(19) United States
(12) Patent Application Publication
(10) Pub. No.: US 2006/0014183 A1
(43) Pub. Date: Jan. 19, 2006

(54) Extendable probes

(76) Inventors: Henrik M. Pfundheller, Horsholm (DK); Michael Meldgaard, Hillerod (DK); Peter Mouritzen, Jyllinge (DK); Peter Stein Nielsen, Birkerod (DK); Mikkel Norholm, Kobenhavn V (DK); Soren Morgenthaler Echwald, Humlebaek (DK)

Correspondence Address: CLARK & ELBING LLP, 101 FEDERAL STREET, BOSTON, MA, 02110, US

(21) Appl. No.: 11/149,910

(22) Filed: Jun. 10, 2005

Related U.S. Application Data
(60) Provisional application No. 60/578,696, filed on Jun. 10, 2004.

(51) Int. Cl. C12Q 1/68; C07H 21/04; C12P 19/34

(52) U.S. Cl. 435/6; 435/91.2; 536/24.3

(57) ABSTRACT

The invention relates to probes which are extendable useful as PCR probes and in probe libraries. The invention further relates to prevention of replication of a primer extension product in PCR reactions.

### CROSS-REFERENCE TO RELATED APPLICATION

This application claims benefit of U.S. Provisional Application No. 60/578,696, filed Jun. 10, 2004, which is hereby incorporated by reference.

### BACKGROUND OF THE INVENTION

The invention relates to probes which are useful as PCR probes and in probe libraries.

The invention further relates to probes and methods for prevention of replication of a primer extension product in PCR reactions.

EP543942 discloses a process for the detection of a target nucleic acid sequence in a sample, said process comprising the preparation of a dual labelled probe and a process using this probe as an improvement over known PCR detection methods.

However the experimental part of the invention disclosed in EP 543942 is restricted to probes having a 3′-PO₄ instead of a 3′-OH in order to block any extension by Taq Polymerase.

Generally the insertion of a phosphate group in the 3′ end of the probe used in PCR analysis is used to prohibit the incorporation of the probe into a primer extension product.

With the advent of microarrays for profiling the expression of thousands of genes, such as GeneChip™ arrays (Affymetrix, Inc., Santa Clara, Calif.), correlations between expressed genes and cellular phenotypes may be identified at a fraction at the cost and labour necessary for traditional methods, such as Northern- or dot-blot analysis. Microarrays permit the development of multiple parallel assays for identifying and validating biomarkers of disease and drug targets which can be used in diagnosis and treatment. Gene expression profiles can also be used to estimate and predict metabolic and toxicological consequences of exposure to an agent (e.g., such as a drug, a potential toxin or carcinogen, etc.) or a condition (e.g., temperature, pH, etc).

Microarray experiments often yield redundant data, only a fraction of which has value for the experimenter. Additionally, because of the highly parallel format of microarray-based assays, conditions may not be optimal for individual capture probes. For these reasons, microarray experiments are most often followed up by, or sequentially replaced by, confirmatory studies using single-gene homogeneous assays. These are most often quantitative PCR-based methods such as the 5′ nuclease assay or other types of dual labelled probe quantitative assays. However, these assays are still time-consuming, single-reaction assays that are hampered by high costs and time-consuming probe design procedures. Further, 5′ nuclease assay probes are relatively large (e.g., 15-30 nucleotides). Thus, the limitations in current homogeneous assay systems create a bottleneck in the validation of microarray findings, and in focused target validation procedures.

An approach to avoid this bottleneck is to omit the expensive dual-labelled indicator probes used in 5′ nuclease assay procedures and molecular beacons and instead use non-sequence-specific DNA intercalating dyes such as SYBR Green that fluoresce upon binding to double-stranded but not single-stranded DNA. Using such dyes, it is possible to universally detect any amplified sequence in real-time. However, this technology is hampered by several problems. For example, nonspecific priming during the PCR amplification process can generate un-intentional non-target amplicons that will contribute in the quantification process. Further, interactions between PCR primers in the reaction to form “primer-dimers” are common. Due to the high concentration of primers typically used in a PCR reaction, this can lead to significant amounts of short double-stranded non-target amplicons that also bind intercalating dyes. Therefore, the preferred method of quantifying mRNA by real-time PCR uses sequence-specific detection probes.

One approach for avoiding the problem of random amplification and the formation of primer-dimers is to use generic detection probes that may be used to detect a large number of different types of nucleic acid molecules, while retaining some sequence specificity has been described by Simeonov, et al. (Nucleic Acid Research 30(17): 91, 2002; U.S. Patent Publication 20020197630) and involves the use of a library of probes comprising more than 10% of all possible sequences of a given length (or lengths). The library can include various non-natural nucleobases and other modifications to stabilize binding of probes/primers in the library to a target sequence. Even so, a minimal length of at least 8 bases is required for most sequences to attain a degree of stability that is compatible with most assay conditions relevant for applications such as real time PCR. Because a universal library of all possible 8-mers contains 65,536 different sequences, even the smallest library previously considered by Simeonov, et al. contains more than 10% of all possibilities, i.e. at least 6554 sequences which is impractical to handle and vastly expensive to construct.

From a practical point of view, several factors limit the ease of use and accessibility of contemporary homogeneous assays applications. The problems encountered by users of conventional assay technologies include:
prohibitively high costs when attempting to detect many different genes in a few samples, because the price to purchase a probe for each transcript is high.
the synthesis of labelled probes is time-consuming and often the time from order to receipt from manufacturer is more than 1 week.
user-designed kits may not work the first time and validated kits are expensive on a per assay basis.
it is difficult to test quickly for a new target or to improve probe design iteratively.
the exact probe sequence of commercial validated probes may be unknown for the customer, resulting in problems with evaluation of results and suitability for scientific publication.
when assay conditions or components are obscure it may be impossible to order reagents from alternative source.

The described invention addresses these practical problems and aims to ensure rapid and inexpensive assay development of accurate and specific assays for quantification of gene transcripts.

### SUMMARY OF THE INVENTION

Generally, the insertion of a phosphate group in the 3′ end of the probe in real-time PCR analysis is used to prohibit the incorporation of the probe into a primer extension product. The present invention features labelled probes which are extendable but contain a replication-preventing moiety. FIG. 1 illustrates the prevention of replication by blocking the extension of the reverse primer.

In one aspect, the invention features a labelled oligonucleotide probe including a sequence complementary to a region of a target nucleic acid sequence, wherein the labelled oligonucleotide probe is extendable by a polymerase to allow incorporation of the labelled oligonucleotide into a primer extension product and wherein the replication of all or part of the oligonucleotide probe by a polymerase is prevented. The probe may include a moiety (e.g., LNA, MGB, HEG, intercalator, INA, ENA, dye, or a quencher) that inhibits the replication. The moiety is, for example, disposed between two nucleotide sequences in the probe, e.g., as a linker. In one embodiment, the complement of a part of the labelled oligonucleotide probe is capable of being a template for the oligonucleotide in a PCR reaction. In another embodiment, the complement of a part of the 3′ end of the oligonucleotide probe is capable of being a template for the oligonucleotide in a PCR reaction. In various embodiments, no more than the eight, e.g., no more than the five or three, nucleotides at the 3′ end are capable of being replicated. At least a part of the labelled oligonucleotide probe may not act as a template for polymerase replication in a reaction which otherwise is capable of generating partially or entirely complementary target sequences for the labelled oligonucleotide probe or may not act as a template for polymerase replication in a reaction which otherwise is capable of generating a complementary part of the labelled oligonucleotide probe sufficient to act as template for the labelled oligonucleotide probe in a PCR reaction. In another embodiment, a substantial part (e.g., 1, 2, 3, 4, 5, 6, 7, 8, 9, or 10 nucleotides) of the 3′ end of the labelled oligonucleotide probe cannot act as a template for polymerase replication in a reaction which otherwise is capable of generating additional partially or entirely complementary probe target sequences sufficient to act as template for the labelled oligonucleotide probe in a PCR reaction. Labelled oligonucleotide probes of the invention may contain two labels, e.g., to generate a detectable signal or to quench a detectable signal. The labelled oligonucleotide probe may also contain a site that is cleavable by a nuclease, e.g., the 5′ to 3′ nuclease activity of a nucleic acid polymerase. Cleavage by the nuclease may further remove a label from the probe, e.g., separate two labels, and lead to an increase or decrease in the amount of a detectable signal produced by the labelled probe. Labelled oligonucleotide probes may also include any naturally occurring or non-naturally occurring nucleotide or other monomers, as described herein. For example, the labelled oligonucleotide probe may include a block of LNA monomers, i.e., two or more LNA monomers in sequence.

The invention further features a polymerase chain reaction (PCR) amplification process for detecting a target nucleic acid sequence in a sample including contacting the sample with at least one labelled oligonucleotide probe of the invention and a first oligonucleotide primer having a sequence complementary to a region in one strand of the target nucleic acid sequence and priming the synthesis of a complementary DNA strand, wherein the first oligonucleotide primer anneals to its complementary region upstream of any labelled oligonucleotide probe annealed to the same nucleic acid strand; amplifying the target nucleic acid sequence using a nucleic acid polymerase having 5′ to 3′ nuclease activity as a template-dependent polymerizing agent under conditions which are permissive for PCR cycling steps of (i) annealing of the first oligonucleotide primer and the labelled oligonucleotide probe to a template nucleic acid sequence contained within the target sequence, and (ii) extending the first oligonucleotide primer wherein the nucleic acid polymerase synthesizes a primer extension product while the 5′ to 3′ nuclease activity of the nucleic acid polymerase simultaneously releases labelled fragments from the annealed duplexes including the labelled oligonucleotide and its complementary template nucleic acid sequence, thereby creating detectable labelled fragments; and detecting the presence or absence of labelled fragments to determine the presence or absence of the target sequence in the sample.

The process may further include providing a second oligonucleotide primer including a sequence complementary to a region in the second strand of the target nucleic acid sequence (i.e., the strand complementary to that which the first primer binds) and priming the synthesis of a complementary DNA strand, wherein the labelled oligonucleotide probe anneals to the target nucleic acid sequence bounded by the first and second oligonucleotide primers. The labelled oligonucleotide probe may include a pair of labels effectively positioned on the oligonucleotide to generate a detectable signal, the labels being separated by a site within the oligonucleotide that is cleaved by the 5′ to 3′ nuclease activity of the nucleic acid polymerase employed. In an alternative embodiment, the labelled oligonucleotide probe includes a pair of labels effectively positioned on the oligonucleotide to quench the generation of detectable signal, the labels being separated by a site within the oligonucleotide that is cleaved by the 5′ to 3′ nuclease activity of the nucleic acid polymerase employed.

In another aspect, the invention features a library of a plurality of labelled oligonucleotide probes, as described herein, wherein each probe in the library includes a recognition sequence tag and a detection moiety, wherein at least one monomer in each oligonucleotide probe is a modified monomer analogue, increasing the binding affinity for the complementary target sequence relative to the corresponding unmodified oligodeoxyribonucleotide, such that the probes have sufficient stability for sequence-specific binding and detection of a substantial fraction of a target nucleic acid in any given target population. In various embodiments, the number of different recognition sequences include less than 10% of all possible sequence tags of a given length(s).

The invention also features a kit containing one or more labelled oligonucleotide probes of the invention and additional components, as described herein.

The labelled oligonucleotide probes of the invention may also be used as multi-probes. It is also desirable to be able to quantify the expression of most genes (e.g., >98%) in the human transcriptome using a limited number of oligonucleotide detection probes in a homogeneous assay system. The present invention solves the problems faced by contemporary approaches to homogeneous assays outlined above by providing a method for construction of generic multi-probes with sufficient sequence specificity—so that they are unlikely to detect a randomly amplified sequence fragment or primer-dimers—but are still capable of detecting many different target sequences each. Such probes are usable in different assays and may be combined in small probe libraries (50 to 500 probes) that can be used to detect and/or quantify individual components in complex mixtures composed of thousands of different nucleic acids (e.g. detecting individual transcripts in the human transcriptome composed of >30,000 different nucleic acids.) when combined with a target specific primer set.

Each multi-probe comprises two elements: 1) a detection element or detection moiety consisting of one or more labels to detect the binding of the probe to the target; and 2) a recognition element or recognition sequence tag ensuring the binding to the specific target(s) of interest. The detection element can be any of a variety of detection principles used in homogeneous assays. The detection of binding is either direct by a measurable change in the properties of one or more of the labels following binding to the target (e.g. a molecular beacon type assay with or without stem structure) or indirect by a subsequent reaction following binding (e.g. cleavage by the 5′ nuclease activity of the DNA polymerase in 5′ nuclease assays).

The recognition element is a novel component of the present invention. It comprises a short oligonucleotide moiety whose sequence has been selected to enable detection of a large subset of target nucleotides in a given complex sample mixture. The novel probes designed to detect many different target molecules are referred to as multi-probes. The concept of designing a probe for multiple targets and exploiting the recurrence of a short recognition sequence by selecting the most frequently encountered sequences is novel and contrary to conventional probes that are designed to be as specific as possible for a single target sequence. The surrounding primers and the choice of probe sequence in combination subsequently ensure the specificity of the multi-probes. The novel design principles arising from attempts to address the largest number of targets with the smallest number of probes are likewise part of the invention. This aspect is enabled by the discovery that very short 8-9 mer LNA mix-mer probes are compatible with PCR based assays. In one aspect of the present invention modified or analogue nucleobases, nucleosidic bases or nucleotides are incorporated in the recognition element, possibly together with minor groove binders and other modifications, that all aim to stabilize the duplex formed between the probe and the target molecule so that the shortest possible probe sequence with the widest range of targets can be used. In a preferred aspect of the invention the modifications are incorporations of LNA residues to reduce the length of the recognition element to 8 or 9 nucleotides while maintaining sufficient stability of the formed duplex to be detectable under ordinary assay conditions.

Preferably, the multi-probes are modified in order to increase the binding affinity of the probe for a target sequence by at least two-fold compared to a probe of the same sequence without the modification, under the same conditions for detection, e.g., such as PCR conditions, or stringent hybridization conditions. The preferred modifications include, but are not limited to, inclusion of nucleobases, nucleosidic bases or nucleotides that have been modified by a chemical moiety or replaced by an analogue to increase the binding affinity. The preferred modifications may also include attachment of duplex stabilizing agents e.g., such as minor-groove-binders (MGB) or intercalating nucleic acids (INA). Additionally the preferred modifications may also include addition of non-discriminatory bases e.g., such as 5-nitroindole, which are capable of stabilizing duplex formation regardless of the nucleobase at the opposing position on the target strand. Finally, multi-probes composed of a non-sugar-phosphate backbone, e.g., PNA, that are capable of binding sequence specifically to a target sequence are also considered modified. All the different binding affinity increased modifications mentioned above will in the following be referred to as “the stabilizing modification(s)”, and the ensuing multi-probe will in the following also be referred to as “modified oligonucleotide”. More preferably the binding affinity of the modified oligonucleotide is at least about 3-fold, 4-fold, 5-fold, or 20-fold higher than the binding of a probe of the same sequence but without the stabilizing modification(s).

Most preferably, the stabilizing modification(s) is inclusion of one or more LNA nucleotide analogs. Probes of from 6 to 12 nucleotides according to the invention may comprise from 1 to 8 stabilizing nucleotides, such as LNA nucleotides. When at least two LNA nucleotides are included, these may be consecutive or separated by one or more non-LNA nucleotides. In one aspect, LNA nucleotides are alpha and/or xylo LNA nucleotides.

The invention also provides oligomer multi-probe library useful under conditions used in NASBA based assays.

NASBA is a specific, isothermal method of nucleic acid amplification suited for the amplification of RNA. Nucleic acid isolation is achieved via lysis with guanidine thiocyanate plus Triton X-100 and ending with purified nucleic acid being eluted from silicon dioxide particles.

Amplification by NASBA involves the coordinated activities of three enzymes, AMV Reverse Transcriptase, RNase H, and T7 RNA Polymerase. Quantitative detection is achieved by way of internal calibrators, added at isolation, which are co-amplified and subsequently identified along with the wild type of RNA using electro chemiluminescence.

The invention also provides an oligomer multi-probe library comprising multi-probes comprising at least one with stabilizing modifications as defined above. Preferably, the probes are less than about 20 nucleotides in length and more preferably less than 12 nucleotides, and most preferably about 8 or 9 nucleotides. Also, preferably, the library comprises less than about 3000 probes and more preferably the library comprises less than 500 probes and most preferably about 100 probes. The libraries containing labelled multi-probes may be used in a variety of applications depending on the type of detection element attached to the recognition element. These applications include, but are not limited to, dual or single labelled assays such as 5′ nuclease assay, molecular beacon applications (see, e.g., Tyagi and Kramer Nat. Biotechnol. 14: 303-308,1996) and other FRET-based assays.

In one aspect of the invention the multi-probes described are designed together to complement each other as a predefined subset of all possible sequences of the given lengths selected to be able to detect/characterize/quantify the largest number of nucleic acids in a complex mixture using the smallest number of multi-probe sequences. These predesigned small subsets of all possible sequences constitute a multi-probe library. The multi-probe libraries described by the present invention attains this functionality at a greatly reduced complexity by deliberately selecting the most commonly occurring oligomers of a given length or lengths while attempting to diversify the selection to get the best possible coverage of the complex nucleic acid target population. In one preferred aspect, probes of the library hybridize with more than about 60% of a target population of nucleic acids, such as a population of human mRNAs. More preferably, the probes hybridize with greater than 70%, greater than 80%, greater than 90%, greater than 95% and even greater than 98% of all target nucleic acid molecules in a population of target molecules.

In a most preferred aspect of the invention, a probe library (i.e., such as about 100 multi-probes) comprising about 0.1% of all possible sequences of the selected probe length(s), is capable of detecting, classifying, and/or quantifying more than 98% of mRNA transcripts in the transcriptome of any specific species, particularly mammals and more particular humans (i.e., >35,000 different mRNA sequences).

The problems with existing homogeneous assays mentioned above are addressed by the use of a multi-probe library according to the invention consisting of a minimal set of short detection probes selected so as to recognize or detect a majority of all expressed genes in a given cell type from a given organism. In one aspect, the library comprises probes that detect each transcript in a transcriptome of greater than about 10,000 genes, greater than about 15,000 genes, greater than about 20,000 genes, greater than about 25,000 genes, greater than about 30,000 genes or greater than about 35,000 genes or equivalent numbers of different mRNA transcripts. In one preferred aspect, the library comprises probes that detect mammalian transcripts sequences, e.g., such as mouse, rat, rabbit, monkey, or human sequences.

By providing a cost efficient multi-probe set useful for rapid development of quantitative real-time and end-point PCR assays, the present invention overcomes the limitations discussed above for contemporary homogeneous assays. The detection element of the multi-probes according to the invention may be single or doubly labelled (e.g., by comprising a label at each end of the probe, or an internal position). Thus, probes according to the invention can be adapted for use in 5′ nuclease assays, molecular beacon assays, FRET assays, and other similar assays. In one aspect, the detection multi-probe comprises two labels capable of interacting with each other to produce a signal or to modify a signal, such that a signal or a change in a signal may be detected when the probe hybridizes to a target sequence. A particular aspect is when the two labels comprise a quencher and a reporter molecule.

In another aspect, the probe comprises a target-specific recognition segment capable of specifically hybridizing to a plurality of different nucleic acid molecules comprising the complementary recognition sequence. A particular detection aspect of the invention referred to as a “molecular beacon with a stem region” is when the recognition segment is flanked by first and second complementary hairpin-forming sequences which may anneal to form a hairpin. A reporter label is attached to the end of one complementary sequence and a quenching moiety is attached to the end of the other complementary sequence. The stem formed when the first and second complementary sequences are hybridized (i.e., when the probe recognition segment is not hybridized to its target) keeps these two labels in close proximity to each other, causing a signal produced by the reporter to be quenched by fluorescence resonance energy transfer (FRET). The proximity of the two labels is reduced when the probe is hybridized to a target sequence and the change in proximity produces a change in the interaction between the labels. Hybridization of the probe thus results in a signal (e.g., fluorescence) being produced by the reporter molecule, which can be detected and/or quantified.

In another aspect, the multi-probe comprises a reporter and a quencher molecule at opposing ends of the short recognition sequence, so that these moieties are in sufficient proximity to each other, that the quencher substantially reduces the signal produced by the reporter molecule. This is the case both when the probe is free in solution as well as when it is bound to the target nucleic acid. A particular detection aspect of the invention referred to as a “5′ nuclease assay” is when the multi-probe may be susceptible to cleavage by the 5′ nuclease activity of the DNA polymerase. This reaction may result in separation of the quencher molecule from the reporter molecule and the production of a detectable signal. Thus, such probes can be used in amplification-based assays to detect and/or quantify the amplification process for a target nucleic acid.

The invention relates to a library of oligonucleotide probes wherein each probe in the library consists of a recognition sequence tag and a detection moiety wherein at least one monomer in each oligonucleotide probe is a modified monomer analogue, increasing the binding affinity for the complementary target sequence relative to the corresponding unmodified oligodeoxyribonucleotide, such that the library probes have sufficient stability for sequence-specific binding and detection of a substantial fraction of a target nucleic acid in any given target population and wherein the number of different recognition sequences comprises less than 10% of all possible sequence tags of a given length(s).

The invention further relates to a library of oligonucleotide probes wherein the recognition sequence tag segment of the probes in the library have been modified in at least one of the following ways:
i) substitution with at least one non-naturally occurring nucleotide
ii) substitution with at least one chemical moiety to increase the stability of the probe.

Further, the invention relates to a library of oligonucleotide probes wherein the recognition sequence tag has a length of 6 to 12 nucleotides, and wherein the preferred length is 8 or 9 nucleotides.

Further, the invention relates to recognition sequence tags that are substituted with LNA nucleotides and wherein more than 90% of the oligonucleotide probes can bind and detect at least two complementary target sequences in a nucleic acid population.

Also preferably, the probe is capable of detecting more than one target in a target population of nucleic acids, e.g., the probe is capable of hybridizing to a plurality of different nucleic acid molecules contained within the target population of nucleic acids.

The invention also provides a method, system and computer program embedded in a computer readable medium (“a computer program product”) for designing multi-probes comprising at least one stabilizing nucleobase. The method comprises querying a database of target sequences (e.g., such as a database of expressed sequences) and designing a small set of probes (e.g., such as 50 or 100 or 200 or 300 or 500) which: i) has sufficient binding stability to bind their respective target sequence under PCR conditions, ii) have limited propensity to form duplex structures with itself, and iii) are capable of binding to and detecting/quantifying at least about 60%, at least about 70%, at least about 80%, at least about 90% or at least about 95% of all the sequences in the given database of sequences, such as a database of expressed sequences.

Probes are designed in silico, which comprise all possible combinations of nucleotides of a given length forming a database of virtual candidate probes. These virtual probes are queried against the database of target sequences to identify probes that comprise the maximal ability to detect the most different target sequences in the database (“optimal probes”). Optimal probes so identified are removed from the virtual probe database. Additionally, target nucleic acids, which were identified by the previous set of optimal probes, are subtracted from the target nucleic acid database. The remaining probes are then queried against the remaining target sequences to identify a second set of optimal probes. The process is repeated until a set of probes is identified which can provide the desired coverage of the target sequence database. The set may be stored in a database as a source of sequences for transcriptome analysis. Multi-probes may be synthesized having recognition sequences, which correspond to those in the database to generate a library of multi-probes.

In one preferred aspect, the target sequence database comprises nucleic acid sequences corresponding to human mRNA (e.g., mRNA molecules, cDNAs, and the like).

In another aspect, the method further comprises calculating stability based on the assumption that the recognition sequence comprises at least one stabilizing nucleotide, such as an LNA molecule. In one preferred aspect the calculated stability is used to eliminate probe recognition sequences with inadequate stability from the database of virtual candidate probes prior to the initial query against the database of target sequence to initiate the identification of optimal probe recognition sequences.

In another aspect, the method further comprises calculating the propensity for a given probe recognition sequence to form a duplex structure with itself based on the assumption that the recognition sequence comprises at least one stabilizing nucleotide, such as an LNA molecule. In one preferred aspect the calculated propensity is used to eliminate probe recognition sequences that are likely to form probe duplexes from the database of virtual candidate probes prior to the initial query against the database of target sequence to initiate the determination of optimal probe recognition sequences.

In another aspect, the method further comprises evaluating the general applicability of a given candidate probe recognition sequence for inclusion in the growing set of optimal probe candidates by both a query against the remaining target sequences as well as a query against the original set of target sequences. In one preferred aspect only probe recognition sequences that are frequently found in both the remaining target sequences and in the original target sequences are added to the growing set of optimal probe recognition sequences. In a most preferred aspect this is accomplished by calculating the product of the scores from these queries and selecting the probes recognition sequence with the highest product that still is among the probe recognition sequences with 20% best score in the query against the current targets.

The invention also provides a computer program embedded in a computer readable medium comprising instructions for searching a database comprising a plurality of different target sequences and for identifying a set of probe recognition sequences capable of identifying to at least about 60%, about 70%, about 80%, about 90% and about 95% of the sequences within the database. In one aspect, the program provides instructions for executing the method described above. In another aspect, the program provides instructions for implementing an algorithm. The invention further provides a system wherein the system comprises a memory for storing a database comprising sequence information for a plurality of different target sequences and also comprises an application program for executing the program instructions for searching the database for a set of probe recognition sequences which is capable of hybridizing to at least about 60%, about 70%, about 80%, about 90% and about 95% of the sequences within the database.

Another aspect of the invention relates to an oligonucleotide probe comprising a detection element and a recognition segment each independently having a length of about 1 to 8 nucleotides, wherein some or all of the nucleotides in the oligonucleotides are substituted by non-natural bases or base analogues having the effect of increasing binding affinity compared to natural nucleobases and/or some or all of the nucleotide units of the oligonucleotide probe are modified with a chemical moiety or replaced by an analogue to increase binding affinity, and/or where said oligonucleotides are modified with a chemical moiety or is an oligonucleotide analogue to increase binding affinity, such that the probe has sufficient stability for binding to the target sequence under conditions suitable for detection, and wherein the probe is capable of detecting more than one complementary target in a target population of nucleic acids.

A preferred embodiment of the invention is a kit for the characterization or detection or quantification of target nucleic acids comprising samples of a library of multi-probes. In one aspect, the kit comprises in silico protocols for their use. In another aspect, the kit comprises information relating to suggestions for obtaining inexpensive DNA primers. The probes contained within these kits may have any or all of the characteristics described above. In one preferred aspect, a plurality of probes comprises at least one stabilizing nucleotide, such as an LNA nucleotide. In another aspect, the plurality of probes comprises a nucleotide coupled to or stably associated with at least one chemical moiety for increasing the stability of binding of the probe. In a further preferred aspect, the kit comprises about 100 different probes. The kits according to the invention allow a user to quickly and efficiently develop an assay for thousands of different nucleic acid targets.

The invention further provides a multi-probe comprising one or more LNA nucleotide, which has a reduced length of about 8, or 9 nucleotides. By selecting commonly occurring 8 and 9-mers as targets it is possible to detect many different genes with the same probe. Each 8 or 9-mer probe can be used to detect more than 7000 different human mRNA sequences. The necessary specificity is then ensured by the combined effect of inexpensive DNA primers for the target gene and by the 8 or 9-mer probe sequence targeting the amplified DNA.

In a preferred embodiment the present invention relates to an oligonucleotide multi-probe library comprising LNA-substituted octamers and nonamers of less than about 1000 sequences, preferably less than about 500 sequences, or more preferably less than about 200 sequences, such as consisting of about 100 different sequences selected so that the library is able to recognize more than about 90%, more preferably more than about 95% and more preferably more than about 98% of mRNA sequences of a target organism or target organ.

A recurring problem in designing real-time PCR detection assays for multiple genes is that the success-rate of these de-novo designs is less than 100%. Troubleshooting a nonfunctional assay can be cumbersome since ideally, a target specific template is needed for each probe, to test the functionality of the detection probe. Furthermore, a target specific template can be useful as a positive control if it is unknown whether the target is available in the test sample. When operating with a limited number of detection probes in a probe library kit as described in the present invention (e.g., 90), it is feasible to also provide positive control targets in the form of PCR-amplifiable templates containing all possible targets for the limited number of probes (e.g., 90). This feature allows users to evaluate the function of each probe, and is not feasible for non-recurring probe-based assays, and thus constitutes a further beneficial feature of the invention. For the suggested preferred probe recognition sequences, we have designed concatamers of control sequences for all probes, containing a PCR-amplifiable target for every probe in the 40 first probes.

Other features and advantages of the invention will be apparent from the following description and the claims.

### DEFINITIONS

The following definitions are provided for specific terms, which are used in the disclosure of the present invention:

As used herein, the term “transcriptome” refers to the complete collection of transcribed elements of the genome of any species.

In addition to mRNAs, it also represents non-coding RNAs which are used for structural and regulatory purposes.

As used herein, the term “replication” is defined as the process of template DNA replication, where a molecule of a DNA polymerase binds to one strand of the DNA and begins moving along it in the 3′ to 5′ direction (of the template strand) using it as a template for assembling by incorporation of nucleoside-triphosphates, a copy of the original strand, synthesized in the 5′ to 3′ direction (of the new strand). Thus the replicated strand will comprise the reverse, complement sequence of the template strand. DNA replication as employed in the PCR reaction is initiated at and extended from the 3′ terminal nucleotide of a oligonucleotide primer annealed to the DNA template strand.

As used herein the term “replication preventing moiety” is defined as a moiety contained in a nucleotide template which will prevent the process of replication of said template. As an example hexaethylene glycol or hexaethylene oxide (HEG) is a non-coding, hydrophilic monomer with many uses. HEG incorporated in the 3′-end of an oligonucleotide probe will prevent extension if the probe is present in a PCR reaction. Also if a PCR primer has a HEG monomer in the middle of its DNA sequence the replication (and hence PCR reaction) will copy up to the HEG but not past it. Therefore a double stranded PCR product using one primer containing a HEG monomer will have a single stranded tail (5′-overlap). In some contexts this is referred to as a PCR stopper.

As used herein, the term “amplicon” refers to small, replicating DNA fragments.

As used herein, a “sample” refers to a sample of tissue or fluid isolated from an organism or organisms, including but not limited to, skin, plasma, serum, spinal fluid, lymph fluid, synovial fluid, urine, tears, blood cells, organs, tumors, and also to samples of in vitro cell culture constituents (including but not limited to conditioned medium resulting from the growth of cells in cell culture medium, recombinant cells and cell components).

By the term “SBC nucleobases” is meant “Selective Binding Complementary” nucleobases, i.e., modified nucleobases that can make stable hydrogen bonds to their complementary nucleobases, but are unable to make stable hydrogen bonds to other SBC nucleobases.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated nucleotide sequence. “Corresponding” means identical to or complementary to the designated sequence.

The oligonucleotide is not necessarily physically derived from any existing or natural sequence but may be generated in any manner, including chemical synthesis, DNA replication, reverse transcription or a combination thereof. The terms “oligonucleotide” or “nucleic acid” intend a polynucleotide of genomic DNA or RNA, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature.

Because mononucleotides are reacted to make oligonucleotides in a manner such that the 5′ phosphate of one mononucleotide pentose ring is attached to the 3′ oxygen of its neighbour in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends.

When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

The term “primer” may refer to more than one primer and refers to an oligonucleotide, whether occurring naturally, as in a purified restriction digest, or produced synthetically, which is capable of acting as a point of initiation of synthesis along a complementary strand when placed under conditions in which synthesis of a primer extension product which is complementary to a nucleic acid strand is catalyzed. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification.

As used herein, the terms “PCR reaction”, “PCR amplification”, “PCR” and “real-time PCR”, also designated RT-PCR are terms used to signify use of various nucleic acid amplification system, which multiplies the target nucleic acids being detected. Examples of such systems include the polymerase chain reaction (PCR) system, quantitative PCR (qPCR) and the ligase chain reaction (LCR) system. Other methods recently described and known to the person of skill in the art are the nucleic acid sequence based amplification (NASBA™, Cangene, Mississauga, Ontario) and Q Beta Replicase systems. The products formed by said amplification reaction may be monitored in real time or after the reaction as an end point measurement.

The complement of a nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the base pairs have disassociated.

As used herein, the term “probe” refers to a labelled oligonucleotide, which forms a duplex structure with a sequence in the target nucleic acid, due to complementarity of at least one sequence in the probe with a sequence in the target region. The probe, preferably, does not contain a sequence complementary to sequence(s) used to prime the polymerase chain reaction.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like.

As defined herein, “5′→3′ nuclease activity” or “5′ to 3′ nuclease activity” refers to that activity of a template-specific nucleic acid polymerase including either a 5′→3′ exonuclease activity traditionally associated with some DNA polymerases whereby nucleotides are removed from the 5′ end of an oligonucleotide in a sequential manner, (i.e., E. coli DNA polymerase I has this activity whereas the Klenow fragment does not), or a 5′→3′ endonuclease activity wherein cleavage occurs more than one nucleotide from the 5′ end, or both.

As used herein, the term “thermo stable nucleic acid polymerase” refers to an enzyme which is relatively stable to heat when compared, for example, to nucleotide polymerases from E. coli and which catalyzes the polymerization of nucleosides. Generally, the enzyme will initiate synthesis at the 3′-end of the primer annealed to the target sequence, and will proceed in the 5′-direction along the template, and if possessing a 5′ to 3′ nuclease activity, hydrolyzing or displacing intervening, annealed probe to release both labelled and unlabelled probe fragments or intact probe, until synthesis terminates. A representative thermo stable enzyme isolated from Thermus aquaticus (Taq) is described in U.S. Pat. No. 4,889,818 and a method for using it in conventional PCR is described in Saiki et al., (1988), Science 239:487.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶, N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues and tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859,1990, Cook, Anti-Cancer Drug Design 6: 585-607, 1991, each of which are hereby incorporated by reference in their entirety).

The term “nucleosidic base” or “nucleobase analogue” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art.

By “universal base” is meant a naturally-occurring or desirably a non-naturally occurring compound or moiety that can pair with a natural base (e.g., adenine, guanine, cytosine, uracil, and/or thymine), and that has a Tₘ differential of 15,12, 10, 8, 6, 4, or 2° C. or less as described herein.

By “oligonucleotide,” “oligomer,” or “oligo” is meant a successive chain of monomers (e.g., glycosides of heterocyclic bases) connected via internucleoside linkages. The linkage between two successive monomers in the oligonucleotide consist of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected from hydrogen and C₁₋₄-alkyl, and R″ is selected from C₁₋₆alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P* at the 3′-position, whereas the right-hand side is bound to the 5′-position of a preceding monomer.

By “LNA unit=38 is meant an individual LNA monomer (e.g., an LNA nucleoside or LNA nucleotide) or an oligomer (e.g., an oligonucleotide or nucleic acid) that includes at least one LNA monomer. LNA units as disclosed in WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA units and their method of synthesis also are disclosed in U.S. Pat. No. 6,043,060, U.S. Pat. No. 6,268,490, PCT/JP98/00945, WO 0107455, WO 0100641, WO 9839352, WO 0056746, WO 0056748, WO 0066604, Morita et al., Bioorg. Med. Chem. Lett. 12(1):73-76, 2002; Hakansson et a., Bioorg. Med. Chem. Lett. 11 (7):935-938, 2001; Koshkin et a., J. Org. Chem. 66(25):8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R^{4′} and R^{2′} as shown in formula (I) below together designate —CH₂—O— or —CH₂—CH₂—O— (also designated ENA).

Further preferred LNA monomers are designated “thio-LNA” or “amino-LNA” including bicyclic structures as disclosed in WO 99/14226, wherein the heteroatom in the bridge between R^{4′} and R^{2′} as shown in formula (I) below together designate —CH₂—S—, —CH₂—CH₂—S—, —CH₂—NH— or —CH₂—CH₂—NH—.

By “LNA modified oligonucleotide” or “LNA substituted oligonucleotide” is meant a oligonucleotide comprising at least one LNA monomer of formula (I), described infra, having the below described illustrative examples of modifications:

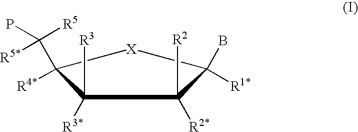

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*)—C(R⁷R⁷*).

B is selected from a modified base as discussed above e.g. an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted indole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R²*, R³, and R³* is a group P* which designates an internucleoside linkage to a preceding monomer, or a 2′/3′-terminal group. The substituents of R¹*, R⁴*, R⁵, R⁵*, R⁶, R⁶*, R⁷, R⁷*, R^{N}, and the ones of R², R²*, R³, and R³* not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C=Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryl-oxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R², R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar term refers to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

The term “Dual labelled probe” refers to an oligonucleotide with two attached labels. In one aspect, one label is attached to the 5′ end of the probe molecule, whereas the other label is attached to the 3′ end of the molecule. A particular aspect of the invention contain a fluorescent molecule attached to one end and a molecule which is able to quench this fluorophore by Fluorescence Resonance Energy Transfer (FRET) attached to the other end. 5′ nuclease assay probes and some Molecular Beacons are examples of Dual labelled probes.

Suitable molecules which is able to quench the fluorophore are compounds disclosed in European Patent Publication EP 1538154. Preferred quenchers are compounds of FIGS. 1 to 9 in said patent publication.

The term “5′ nuclease assay probe” refers to a dual labelled probe which may be hydrolyzed by the 5′-3′ exonuclease activity of a DNA polymerase. A 5′ nuclease assay probes is not necessarily hydrolyzed by the 5′-3′ exonuclease activity of a DNA polymerase under the conditions employed in the particular PCR assay. The name “5′ nuclease assay” is used regardless of the degree of hydrolysis observed and does not indicate any expectation on behalf of the experimenter. The term “5′ nuclease assay probe” and “5′ nuclease assay” merely refers to assays where no particular care has been taken to avoid hydrolysis of the involved probe. “5′ nuclease assay probes” are often referred to as a “TaqMan assay probes”, and the “5′ nuclease assay“as “TaqMan assay”. These names are used interchangeably in this application.

The term “oligonucleotide analogue” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein like backbone. In PNA, nucleobases are attached to the uncharged polyamide backbone yielding a chimeric pseudopeptide-nucleic acid structure, which is homomorphous to nucleic acid forms.

The term “Molecular Beacon” refers to a single or dual labelled probe which is not likely to be affected by the 5′-3′ exonuclease activity of a DNA polymerase. Special modifications to the probe, polymerase or assay conditions have been made to avoid separation of the labels or constituent nucleotides by the 5′-3′ exonuclease activity of a DNA polymerase. The detection principle thus rely on a detectable difference in label elicited signal upon binding of the molecular beacon to its target sequence. In one aspect of the invention the oligonucleotide probe forms an intramolecular hairpin structure at the chosen assay temperature mediated by complementary sequences at the 5′- and the 3′-end of the oligonucleotide. The oligonucleotide may have a fluorescent molecule attached to one end and a molecule attached to the other, which is able to quench the fluorophore when brought into close proximity of each other in the hairpin structure. In another aspect of the invention, a hairpin structure is not formed based on complementary structure at the ends of the probe sequence instead the detected signal change upon binding may result from interaction between one or both of the labels with the formed duplex structure or from a general change of spatial conformation of the probe upon binding—or from a reduced interaction between the labels after binding. A particular aspect of the molecular beacon contain a number of LNA residues to inhibit hydrolysis by the 5′-3′ exonuclease activity of a DNA polymerase.

The term “multi-probe” as used herein refers to a probe which comprises a recognition segment which is a probe sequence sufficiently complementary to a recognition sequence in a target nucleic acid molecule to bind to the sequence under moderately stringent conditions and/or under conditions suitable for PCR, 5′ nuclease assay and/or Molecular Beacon analysis (or generally any FRET-based method). Such conditions are well known to those of skill in the art. Preferably, the recognition sequence is found in a plurality of sequences being evaluated, e.g., such as a transcriptome. A multi-probe according to the invention may comprise a non-natural nucleotide (“a stabilizing nucleotide”) and may have a higher binding affinity for the recognition sequence than a probe comprising an identical sequence but without the stabilizing modification. Preferably, at least one nucleotide of a multi-probe is modified by a chemical moiety (e.g., covalently or otherwise stably associated with during at least hybridization stages of a PCR reaction) for increasing the binding affinity of the recognition segment for the recognition sequence.

As used herein, a multi-probe with an increased “binding affinity” for a recognition sequence than a probe which comprises the same sequence but which does not comprise a stabilizing nucleotide, refers to a probe for which the association constant (Kₐ) of the probe recognition segment is higher than the association constant of the complementary strands of a double-stranded molecule. In another preferred embodiment, the association constant of the probe recognition segment is higher than the dissociation constant (K_{d}) of the complementary strand of the recognition sequence in the target sequence in a double stranded molecule.

A “multi-probe library” or “library of multi-probes” comprises a plurality of multi-probes, such that the sum of the probes in the library are able to recognise a major proportion of a transcriptome, including the most abundant sequences, such that about 60%, about 70%, about 80%, about 85%, more preferably about 90%, and still more preferably 95%, of the target nucleic acids in the transcriptome, are detected by the probes.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, inosine with C, pseudoisocytosine with G, etc.

The term “succeeding monomer” relates to the neighboring monomer in the 5′-terminal direction and the “preceding monomer” relates to the neighboring monomer in the 3′-terminal direction.

As used herein, the term “target population” refers to a plurality of different sequences of nucleic acids, for example the genome of a particular species including the transcriptome thereof, wherein the transcriptome refers to the complete collection of transcribed elements of the genome of any species.

As used herein, the term “target nucleic acid” refers to any relevant nucleic acid of a single specific sequence, e. g., a biological nucleic acid, e. g., derived from a patient, an animal (a human or non-human animal), a plant, a bacteria, a fungi, an archae, a cell, a tissue, an organism, etc. For example, where the target nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e. g., a human patient. In this embodiment, the invention optionally further includes selecting a treatment, diagnosing a disease, or diagnosing a genetic predisposition to a disease, based upon detection of the target nucleic acid.

As used herein, the term “target sequence” refers to a specific nucleic acid sequence within any target nucleic acid.

The term “stringent conditions”, as used herein, is the “stringency” which occurs within a range from about Tₘ-5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383,1969; and John, et al. Nature 223: 582-587, 1969.

### BRIEF DESCRIPTION OF THE DRAWINGS

FIG. 1 is a schematic depiction of a labelled oligonucleotide probe of the invention. The labelled probe is extendable, e.g., in a PCR reaction. The modification present in the probe prevent replication of the probe using the reverse primer.

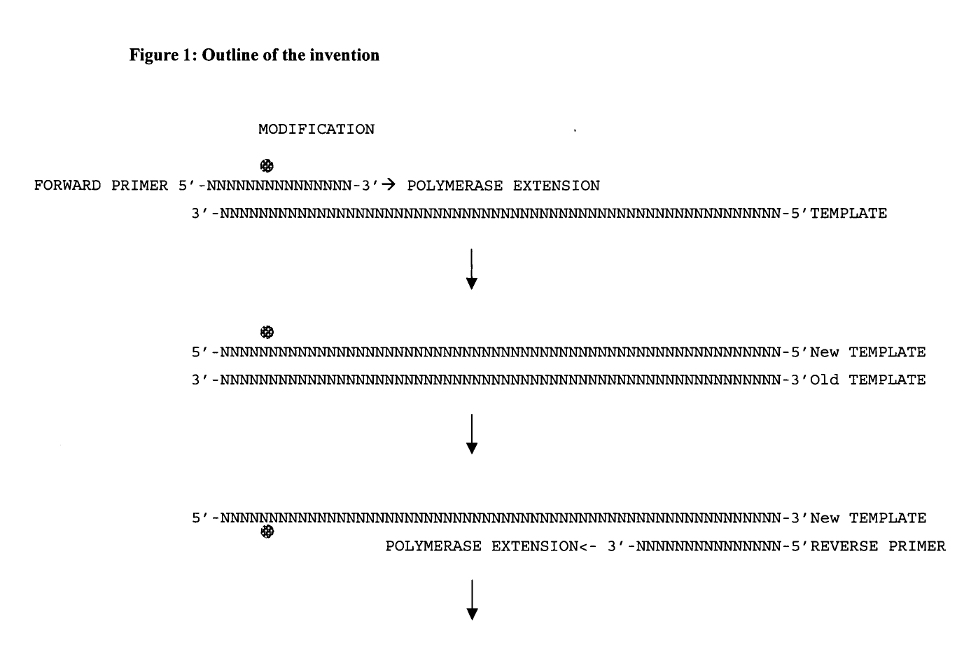

FIGS. 2A and 2B are electrophoresis gels showing the results of primer extension experiments with EQ#16215, 16216, 16221, 16222, 16224, and 16225 (A, gel stained for nucleic acids with GelStar and B, autoradiography of the same gel).

FIGS. 3A and 3B are electrophoresis gels showing the results of primer extension experiments with EQ#16435, 16340, 16342, and 16343 (A, gel stained for nucleic acids with GelStar and B, autoradiography of the same gel).

FIGS. 4A and 4B are electrophoresis gels showing the results of extension experiments with EQ#16214 (A, gel scanned in the Fluorescein-channel immediately after electrophoresis and B, subsequent to GelStar staining).

FIG. 5 is a composite of electrophoresis gels showing the results of extension experiments with EQ#16214, EQ#16221 and EQ#16222.

FIG. 6 is a graph of and increase in intensity from a real time PCR experiment employing EQ#16215.

### DETAILED DESCRIPTION OF THE INVENTION

The invention features labelled oligonucleotide probes, also referred to as extendable probes, and methods of their use. In general, the probes are extendible by a polymerase, but at least a part of the probe is not replicable. The probes may be used to detect the presence or absence of a target sequence in a sample, as described herein. The labelled nature of the probes allows for the detection of the probes in various assays. The probes may be designed to include a nuclease site, or other labile site, to enable cleavage of the probe during as assay, e.g., to cleave the label from the probe or one of a pair of labels, e.g., that interact to generate or quench a detectable signal. Suitable labels are described herein.

The labelled probes typically include a moiety that prevents the process of replication of all or part of the nucleotide sequence that contains the probe, e.g., either the probe itself or an extension product containing a probe. Examples of such moieties include hexaethylene glycol or hexaethylene oxide (HEG), LNA, MGB, intercalator, INA, ENA, dye, and a quencher, as described herein.

The probes may be synthesized by methods known in the art, e.g., as described in WO03020739, WO2004113563, WO2004035819, WO2004020575, WO03095467, WO2004024314, and WO03039523.

The labelled oligonucleotide probes of the invention may be employed in an amplification assay. In such as assay, a labelled probe and one or more primers are contacted with a sample. The primer and probe are designed such that, if a target sequence is present in the sample, the primer and probe anneal, with the probe disposed upstream from the primer. In one example, a polymerase having 5′ to 3′ activity is employed to extend the primer and to cleave the labelled probe. The cleavage generally results in the creation of a detectable signal, e.g., fluorescence. The detectable signal may be generated from the release of one of a pair of labels that interact to quench a signal (i.e., the cleavage increases the amount of a particular signal) or to generate a signal, e.g., from FRET (i.e., the cleavage decreases the amount of a particular signal). Such an assay may be used to identify the presence or absence of a particular target sequence, to quantify the amount of a target sequence, or to track the progression of a particular amplification.

A labelled oligonucleotide probe of the invention may also be used as a multi-probe, e.g., as described in U.S. 2005/0089889, hereby incorporated by reference. A “multi-probe” according to the invention is preferably a short sequence probe which binds to a recognition sequence found in a plurality of different target nucleic acids, such that the multi-probe specifically hybridizes to the target nucleic acid but do not hybridize to any detectable level to nucleic acid molecules which do not comprise the recognition sequence. Preferably, a collection of multi-probes, or multi-probe library, is able to recognize a major proportion of a transcriptome, including the most abundant sequences, such as about 60%, about 70%, about 80%, about 85%, more preferably about 90%, and still more preferably 95%, of the target nucleic acids in the transcriptome, are detected by the probes. A multi-probe according to the invention comprises a “stabilizing modification” e.g. such as a non-natural nucleotide (“a stabilizing nucleotide”) and has higher binding affinity for the recognition sequence than a probe comprising an identical sequence but without the stabilizing sequence. Preferably, at least one nucleotide of a multi-probe is modified by a chemical moiety (e.g., covalently or otherwise stably associated with the probe during at least hybridization stages of a PCR reaction) for increasing the binding affinity of the recognition segment for the recognition sequence.

In one aspect, a multi-probe of from 6 to 12 nucleotides comprises from 1 to 6 or even up to 12 stabilizing nucleotides, such as LNA nucleotides. An LNA enhanced probe library contains short probes that recognize a short recognition sequence (e.g., 8-9 nucleotides). LNA nucleobases can comprise α-LNA molecules (see, e.g., WO 00/66604) or xylo-LNA molecules (see, e.g., WO 00/56748).

In one aspect, it is preferred that the Tₘ of the multi-probe when bound to its recognition sequence is between about 55° C. to about 70° C.

In another aspect, the multi-probes comprise one or more modified nucleobases. Modified base units may comprise a cyclic unit (e.g. a carbocyclic unit such as pyrenyl) that is joined to a nucleic unit, such as a 1′-position of furasonyl ring through a linker, such as a straight of branched chain alkylene or alkenylene group. Alkylene groups suitably having from 1 (i.e., —CH₂—) to about 12 carbon atoms, more typically 1 to about 8 carbon atoms, still more typically 1 to about 6 carbon atoms. Alkenylene groups suitably have one, two or three carbon-carbon double bounds and from 2 to about 12 carbon atoms, more typically 2 to about 8 carbon atoms, still more typically 2 to about 6 carbon atoms.

Multi-probes according to the invention are ideal for performing such assays as real-time PCR as the probes according to the invention are preferably less than about 25 nucleotides, less than about 15 nucleotides, less than about 10 nucleotides, e.g., 8 or 9 nucleotides. Preferably, a multi-probe can specifically hybridize with a recognition sequence within a target sequence under PCR conditions and preferably the recognition sequence is found in at least about 50, at least about 100, at least about 200, at least about 500 different target nucleic acid molecules. A library of multi-probes according to the invention will comprise multi-probes, which comprise non-identical recognition sequences, such that any two multi-probes hybridize to different sets of target nucleic acid molecules. In one aspect, the sets of target nucleic acid molecules comprise some identical target nucleic acid molecules, i.e., a target nucleic acid molecule comprising a gene sequence of interest may be bound by more than one multi-probe. Such a target nucleic acid molecule will contain at least two different recognition sequences which may overlap by one or more, but less than x nucleotides of a recognition sequence comprising x nucleotides.

In one aspect, a multi-probe library comprises a plurality of different multi-probes, each different probe localized at a discrete location on a solid substrate. As used herein, “localize” refers to being limited or addressed at the location such that hybridization event detected at the location can be traced to a probe of known sequence identity. A localized probe may or may not be stably associated with the substrate. For example, the probe could be in solution in the well of a microtiter plate and thus localized or addressed to the well. Alternatively, or additionally, the probe could be stably associated with the substrate such that it remains at a defined location on the substrate after one or more washes of the substrate with a buffer. For example, the probe may be chemically associated with the substrate, either directly or through a linker molecule, which may be a nucleic acid sequence, a peptide or other type of molecule, which has an affinity for molecules on the substrate.

Alternatively, the target nucleic acid molecules may be localized on a substrate (e.g., as a cell or cell lysate or nucleic acids dotted onto the substrate).

Once the appropriate sequences are determined, multi-LNA probes are preferably chemically synthesized using commercially available methods and equipment as described in the art (Tetrahedron 54: 3607-30, 1998). For example, the solid phase phosphoramidite method can be used to produce short LNA probes (Caruthers, et al., Cold Spring Harbor Symp. Quant. Biol. 47:411-418,1982, Adams, et al., J. Am. Chem. Soc. 105: 661 (1983).

The determination of the extent of hybridization of multi-probes from a multi-probe library to one or more target sequences (preferably to a plurality of target sequences) may be carried out by any of the methods well known in the art. If there is no detectable hybridization, the extent of hybridization is thus 0. Typically, labelled signal nucleic acids are used to detect hybridization. Complementary nucleic acids or signal nucleic acids may be labelled by any one of several methods typically used to detect the presence of hybridized polynucleotides. The most common method of detection is the use of ligands, which bind to labelled antibodies, fluorophores or chemiluminescent agents. Other labels include antibodies, which can serve as specific binding pair members for a labelled ligand. The choice of label depends on sensitivity required, ease of conjugation with the probe, stability requirements, and available instrumentation.

LNA-containing-probes are typically labelled during synthesis. The flexibility of the phosphoramidite synthesis approach furthermore facilitates the easy production of LNAs carrying all commercially available linkers, fluorophores and labelling-molecules available for this standard chemistry. LNA may also be labelled by enzymatic reactions e.g. by kinasing.

Multi-probes according to the invention can comprise single labels or a plurality of labels. In one aspect, the plurality of labels comprise a pair of labels which interact with each other either to produce a signal or to produce a change in a signal when hybridization of the multi-probe to a target sequence occurs.

In another aspect, the multi-probe comprises a fluorophore moiety and a quencher moiety, positioned in such a way that the hybridized state of the probe can be distinguished from the unhybridized state of the probe by an increase in the fluorescent signal from the nucleotide. In one aspect, the multi-probe comprises, in addition to the recognition element, first and second complementary sequences, which specifically hybridize to each other, when the probe is not hybridized to a recognition sequence in a target molecule, bringing the quencher molecule in sufficient proximity to said reporter molecule to quench fluorescence of the reporter molecule. Hybridization of the target molecule distances the quencher from the reporter molecule and results in a signal, which is proportional to the amount of hybridization.

In another aspect, where polymerization of strands of nucleic acids can be detected using a polymerase with 5′ nuclease activity. Fluorophore and quencher molecules are incorporated into the probe in sufficient proximity such that the quencher quenches the signal of the fluorophore molecule when the probe is hybridized to its recognition sequence. Cleavage of the probe by the polymerase with 5′ nuclease activity results in separation of the quencher and fluorophore molecule, and the presence in increasing amounts of signal as nucleic acid sequences

In the present context, the term “label” means a reporter group, which is detectable either by itself or as a part of a detection series. Examples of functional parts of reporter groups are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radio isotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy). Especially interesting examples are biotin, fluorescein, Texas Red, rhodamine, dinitrophenyl, digoxigenin, Ruthenium, Europium, Cy5, Cy3, etc.

Suitable samples of target nucleic acid molecule may comprise a wide range of eukaryotic and prokaryotic cells, including protoplasts; or other biological materials, which may harbour target nucleic acids. The methods are thus applicable to tissue culture animal cells, animal cells (e.g., blood, serum, plasma, reticulocytes, lymphocytes, urine, bone marrow tissue, cerebrospinal fluid or any product prepared from blood or lymph) or any type of tissue biopsy (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy, e.g., homogenized in lysis buffer), archival tissue nucleic acids, plant cells or other cells sensitive to osmotic shock and cells of bacteria, yeasts, viruses, mycoplasmas, protozoa, rickettsia, fungi and other small microbial cells and the like.

Target nucleic acids which are recognized by a plurality of multi-probes can be assayed to detect sequences which are present in less than 10% in a population of target nucleic acid molecules, less than about 5%, less than about 1%, less than about 0. 1%, and less than about 0.01% (e.g., such as specific gene sequences). The type of assay used to detect such sequences is a non-limiting feature of the invention and may comprise PCR or some other suitable assay as is known in the art or developed to detect recognition sequences which are found in less than 10% of a population of target nucleic acid molecules.

In one aspect, the assay to detect the less abundant recognition sequences comprises hybridizing at least one primer capable of specifically hybridizing to the recognition sequence but substantially incapable of hybridizing to more than about 50, more than about 25, more than about 10, more than about 5, more than about 2 target nucleic acid molecules (e.g., the probe recognizes both copies of a homozygous gene sequence), or more than one target nucleic acid in a population (e.g., such as an allele of a single copy heterozygous gene sequence present in a sample). In one preferred aspect, a pair of such primers is provided that flank the recognition sequence identified by the multi-probe, i.e., are within an amplifiable distance of the recognition sequence such that amplicons of about 40-5000 bases can be produced, and preferably, 50-500 or more preferably 60-100 base amplicons are produced. One or more of the primers may be labelled.

Various amplifying reactions are well known to one of ordinary skill in the art and include, but are not limited to PCR, RT-PCR, LCR, in vitro transcription, rolling circle PCR, OLA and the like. Multiple primers can also be used in multiplex PCR for detecting a set of specific target molecules.

In one aspect, a plurality of n-mers of n nucleotides is generated in silico, containing all possible n-mers. A subset of n-mers are selected which have a Tₘ≧60° C. In another aspect, a subset of these probes is selected which do not self-hybridize to provide a list or database of candidate n-mers. The sequence of each n-mer is used to query a database comprising a plurality of target sequences. Preferably, the target sequence database comprises expressed sequences, such as human mRNA sequences.

From the list of candidate n-mers used to query the database, n-mers are selected that identify a maximum number of target sequences (e.g., n-mers which comprise recognition segments which are complementary to subsequences of a maximal number of target sequences in the target database) to generate an n-mer/target sequence matrix. Sequences of n-mers, which bind to a maximum number of target sequences, are stored in a database of optimal probe sequences and these are subtracted from the candidate n-mer database. Target sequences that are identified by the first set of optimal probes are removed from the target sequence database. The process is then repeated for the remaining candidate probes until a set of multi-probes is identified comprising n-mers which cover more than about 60%, more than about 80%, more than about 90% and more than about 95% of target sequences. The optimal sequences identified at each step may be used to generate a database of virtual multi-probes sequences. Multi-probes may then be synthesized which comprise sequences from the multi-probe database.

In another aspect, the method further comprises evaluating the general applicability of a given candidate probe recognition sequence for inclusion in the growing set of optimal probe candidates by both a query against the remaining target sequences as well as a query against the original set of target sequences. In one preferred aspect only probe recognition sequences that are frequently found in both the remaining target sequences and in the original target sequences are added to in the growing set of optimal probe recognition sequences. In a most preferred aspect this is accomplished by calculating the product of the scores from these queries and selecting the probes recognition sequence with the highest product that still is among the probe recognition sequences with 20% best score in the query against the current targets.

The invention also provides computer program products for facilitating the method described above. In one aspect, the computer program product comprises program instructions, which can be executed by a computer or a user device connectable to a network in communication with a memory.

The invention further provides a system comprising a computer memory comprising a database of target sequences and an application system for executing instructions provided by the computer program product.

Kits Comprising Multi-Probes

A preferred embodiment of the invention is a kit for the characterisation or detection or quantification of target nucleic acids comprising samples of a library of multi-probes. In one aspect, the kit comprises in silico protocols for their use. In another aspect, the kit comprises information relating to suggestions for obtaining inexpensive DNA primers. The probes contained within these kits may have any or all of the characteristics described above. In one preferred aspect, a plurality of probes comprises a least one stabilizing nucleobase, such as an LNA nucleobase.

In another aspect, the plurality of probes comprises a nucleotide coupled or stably associated with at least one chemical moiety for increasing the stability of binding of the probe. In a further preferred aspect, the kit comprises a number of different probes for covering at least 60% of a population of different target sequences such as a transcriptome. In one preferred aspect, the transcriptome is a human transcriptome.

In another aspect, the kit comprises at least one probe labelled with one or more labels. In still another aspect, one or more probes comprise labels capable of interacting with each other in a FRET-based assay, i.e., the probes may be designed to perform in 5′ nuclease or Molecular Beacon-based assays.

The kits according to the invention allow a user to quickly and efficiently to develop assays for many different nucleic acid targets. The kit may additionally comprise one or more reagents for performing an amplification reaction, such as PCR.

### EXAMPLES

The invention will now be further illustrated with reference to the following examples. It will be appreciated that what follows is by way of example only and that modifications to detail may be made while still falling within the scope of the invention.

In the following Examples probe reference numbers designate the LNA-oligonucleotide sequences shown in the synthesis examples below.

TABLE 1
SEQUENCES

| | | | | |
|---|---|---|---|---|
| EQ | | | | Position |
| Number | Name | Type | Sequence | in gene |

| | | | | |
|---|---|---|---|---|
| 13992 | Dual-labelled- | 5′ nuclease assay probe | 5′-FITC-aaGGAGAAG- | 469-477 |
| | 469 | | Eclipse-3′ | |
| 13994 | Dual-labelled- | 5′ nuclease assay probe | 5′-FITC-cAAGGAAAg- | 570-578 |
| | 570 | | Eclipse-3′ | |
| 13996 | Dual-labelled- | 5′ nuclease assay probe | 5′-FITC-ctGGAGCaG- | 671-679 |
| | 671 | | Eclipse-3′ | |
| 13997 | Beacon-469 | Molecular Beacon | 5′-FITC-CAAGGAGAAGTTG- | |
| | | | Dabcyl -3′ (SEQ ID NO: 1) | |
| 14148 | Beacon-570 | Molecular Beacon | 5′-FITC-CAAGGAAAGttG- | |
| | | | Dabcyl-3′ (SEQ ID NO: 2) | |
| 14165 | SYBR-Probe- | SYBR-Probe | 5′-SYBR101-NH2C6- | |
| | 570 | | cAAGGAAAg-3′ | |
| 14012 | SSA4-469-F | Primer | cgcgtttactttgaaaaattctg | |
| | | | (SEQ ID NO: 3) | |
| 14013 | SSA4-469-R | Primer | gcttccaatttcctggcatc | |
| | | | (SEQ ID NO: 4) | |
| 14014 | SSA4-570-F | Primer | gcccaagatgctataaattggttag | |
| | | | (SEQ ID NO: 5) | |
| 14015 | SSA4-570-R | Primer | gggtttgcaacaccttctagttc | |
| | | | (SEQ ID NO: 6) | |
| 14016 | SSA4-671-F | Primer | tacggagctgcaggtggt | |
| | | | (SEQ ID NO: 7) | |
| 14017 | SSA4-671-R | Primer | gttgggccgttgtctggt | |
| | | | (SEQ ID NO: 8) | |
| 14115 | POL5-469-F | Primer | gcgagagaaaacaagcaagg | |
| | | | (SEQ ID NO: 9) | |
| 14116 | POL5-469-R | Primer | attcgtcttcactggcatca | |
| | | | (SEQ ID NO: 10) | |
| 14117 | APG9-570-F | Primer | cagctaaaaatgatgacaataatgg | |
| | | | (SEQ ID NO: 11) | |
| 14118 | APG9-570-R | Primer | attacatcatgattagggaatgc | |
| | | | (SEQ ID NO: 12) | |
| 14119 | HSP82-671-F | Primer | gggtttgaacattgatgagga | |
| | | | (SEQ ID NO: 13) | |
| 14120 | HSP82-671-R | Primer | ggtgtcagctggaacctctt | |
| | | | (SEQ ID NO: 14) | |

Capitals designate LNA monomers (A, G, mC, T).
Small letters designate DNA monomers (a, g, c, t).
Fitc = Fluorescein; Dabcyl = Dabcyl quencher.

### Example 1

### Synthesis, Deprotection and Purification of Dual Labelled Oligonucleotides

The dual labelled oligonucleotides EQ13992 to EQ14148 (Table 1) were prepared on an automated DNA synthesizer (Expedite 8909 DNA synthesizer, PerSeptive Biosystems, 0.2 μmol scale) using the phosphoramidite approach (Beaucage and Caruthers, Tetrahedron Lett. 22: 1859-1862, 1981) with 2-cyanoethyl protected LNA and DNA phosphoramidites, (Sinha, et al., Tetrahedron Lett.24: 5843-5846, 1983). CPG solid supports derivatized with either eclipse quencher (EQ13992-EQ13996) or dabcyl (EQ13997-EQ14148) and 5′-fluorescein phosphoramidite (GLEN Research, Sterling, Va., USA). The synthesis cycle was modified for LNA phosphoramidites (250 s coupling time) compared to DNA phosphoramidites. 1H-tetazole or 4,5-dicyanoimidazole (Proligo, Hamburg, Germany) was used as activator in the coupling step.

The oligonucleotides were deprotected using 32% aqueous ammonia (1 h at room temperature, then 2 hours at 60° C.) and purified by HPLC (Shimadzu-SpectraChrom series; Xterra™ RP18 column, 10?m 7.8×150 mm (Waters). Buffers: A: 0.05M Triethylammonium acetate pH 7.4. B. 50% acetonitrile in water. Eluent: 0-25 min: 10-80% B; 25-30 min: 80% B).

The composition and purity of the oligonucleotides were verified by MALDI-MS (PerSeptive Biosystem, Voyager DE-PRO) analysis, see Table 2.

TABLE 2

| EQ# | MW (Calc.) | MW (Found) |
|---|---|---|
| 13992 | 4091.8 Da. | 4091.6 Da. |
| 13994 | 4051.9 Da. | 4049.3 Da. |
| 13996 | 4020.8 Da. | 4021.6 Da. |
| 13997 | 5426.3 Da. | 5421.2 Da. |

### Example 2

### Production of cDNA Standards of SSA4 for Detection With 9-mer Probes

The functionality of the constructed 9mer probes were analysed in PCR assays where the probes ability to detect different SSA4 PCR amplicons were questioned. Template for the PCR reaction was cDNA obtained from reverse transcription of cRNA produced from in vitro transcription of a downstream region of the SSA4 gene in the expression vector pTRlampl8 (Ambion). The downstream region of the SSA4 gene was cloned as follows:

PCR Amplification

Amplification of the partial yeast gene was done by standard PCR using yeast genomic DNA as template. Genomic DNA was prepared from a wild type standard laboratory strain of Saccharomyces cerevisiae using the Nucleon MiY DNA extraction kit (Amersham Biosciences) according to supplier's instructions. In the first step of PCR amplification, a forward primer containing a restriction enzyme site and a reverse primer containing a universal linker sequence were used. In this step 20 bp was added to the 3′-end of the amplicon, next to the stop codon. In the second step of amplification, the reverse primer was exchanged with a nested primer containing a poly-T₂₀ tail and a restriction enzyme site. The SSA4 amplicon contains 729 bp of the SSA4 ORF plus a 20 bp universal linker sequence and a poly-A₂₀ tail.

The PCR primers used were (SEQ ID NOs: 15-17):

| | |
|---|---|
| YER103W-For-Sacl: | acgtgagctcattgaaactgcaggtggt |
| | attatga |
| YER103W-Rev-Uni: | gatccccgggaattgccatgctaatcaacctc |
| | ttcaaccgttgg |
| Uni-polyT-BamHI: | acgtggatccttttttttttttttttttttga |
| | tccccgggaattgccatg. |

Plasmid DNA Constructs

The PCR amplicon was cut with the restriction enzymes, EcoRI+BamHI. The DNA fragment was ligated into the pTRIamp18 vector (Ambion) using the Quick Ligation Kit (New England Biolabs) according to the supplier's instructions and transformed into E. coli DH-5 by standard methods.

DNA Sequencing

To verify the cloning of the PCR amplicon, plasmid DNA was sequenced using M13 forward and M13 reverse primers and analysed on an ABI 377.

In Vitro Transcription

SSA4 cRNA was obtained by performing in vitro transcription with the Megascript T7 kit (Ambion) according to the supplier's instructions.

Reverse Transcription

Reverse transcription was performed with 1 μg of cRNA and 0.2 U of the reverse transcriptase Superscript II RT (Invitrogen) according to the suppliers instructions except that 20 U Superase-In (RNAse inhibitor—Ambion) was added. The produced cDNA was purified on a QiaQuick PCR purification column (Qiagen) according to the supplier's instructions using the supplied EB-buffer for elution. The DNA concentration of the eluted cDNA was measured and diluted to a concentration of SSA4 cDNA copies corresponding to 2×10⁷ copies pr μL.

### Example 3

### Protocol for Dual Label Probe Assays

Reagents for the dual label probe PCRs were mixed according to the following scheme (Table 3):

TABLE 3

| Reagents | Final Concentration | |
|---|---|---|
| H₂O | | |
| GeneAmp 10× PCR buffer II | 1× | |
| Mg²⁺ | 5.5 | mM |
| dNTP | 0.2 | mM |
| Dual Label Probe | 0.1 or 0.3 | μM* |
| Template | 1 | μL |
| Forward primer | 0.2 | μM |
| Reverse primer | 0.2 | μM |
| AmpliTaq Gold | 2.5 | U |
| Total | 50 | μL |

*Final concentration of 5′ nuclease assay probe 0.1 μM and Beacon/SYBR-probe 0.3 μM.

In the present experiments 2×10⁷ copies of the SSA4 cDNA was added as template. Assays were performed in a DNA Engine Opticon® (MJ Research) using the following PCR cycle protocols:

TABLE 4

| 5′ nuclease assays | Beacon & SYBR-probe Assays |
|---|---|
| 95° C. for 7 minutes & | 95° C. for 7 minutes & |
| 40 cycles of: | 40 cycles of: |
| 94° C. for 20 seconds | 94° C. for 30 seconds |
| 60° C. for 1 minute | 52° C. for 1 minute* |
| Fluorescence | Fluorescence |
| detection | detection |
| | 72° C. for 30 seconds |

*For the Beacon-570 with 9-mer recognition site the annealing temperature was reduced to 44° C.

The composition of the PCR reactions shown in Table 3 together with PCR cycle protocols listed in Table 4 will be referred to as standard 5′ nuclease assay or standard Beacon assay conditions.

### Example 4

### Specificity of 9-mer 5′ Nuclease Assay Probes

The specificity of the 5′ nuclease assay probes were demonstrated in assays where each of the probes was added to 3 different PCR reactions each generating a different SSA4 PCR amplicon. Each probe only produces a fluorescent signal together with the amplicon it was designed to detect. Importantly the different probes had very similar cycle threshold Cₜ values (from 23.2 to 23.7), showing that the assays and probes have a very equal efficiency. Furthermore it indicates that the assays should detect similar expression levels when used in used in real expression assays. This is an important finding, because variability in performance of different probes is undesirable.

### Example 5

### Specificity of 9 and 10-mer Molecular Beacon Probes

The ability to detect in real time, newly generated PCR amplicons was also demonstrated for the molecular beacon design concept. The Molecular Beacon designed against the 469 amplicon with a 10-mer recognition sequence produced a clear signal when the SSA4 cDNA template and primers for generating the 469 amplicon were present in the PCR, The observed Cₜ value was 24.0 and very similar to the ones obtained with the 5′ nuclease assay probes again indicating a very similar sensitivity of the different probes. No signal was produced when the SSA4 template was not added. A similar result was produced by the Molecular Beacon designed against the 570 amplicon with a 9-mer recognition sequence,

### Example 6

### Specificity of 9-mer SYBR-Probes

The ability to detect newly generated PCR amplicons was also demonstrated for the SYBR-probe design concept. The 9-mer SYBR-probe designed against the 570 amplicon of the SSA4 cDNA produced a clear signal when the SSA4 cDNA template and primers for generating the 570 amplicon were present in the PCR. No signal was produced when the SSA4 template was not added.

### Example 7

### Quantification of Transcript Copy Number

The ability to detect different levels of gene transcripts is an essential requirement for a probe to perform in a true expression assay. The fulfilment of the requirement was shown by the three 5′ nuclease assay probes in an assay where different levels of the expression vector derived SSA4 cDNA was added to different PCR reactions together with one of the 5′ nuclease assay probes. Composition and cycle conditions were according to standard 5′ nuclease assay conditions.

The cDNA copy number in the PCR before start of cycling is reflected in the cycle threshold value Cₜ, i.e., the cycle number at which signal is first detected. Signal is here only defined as signal if fluorescence is five times above the standard deviation of the fluorescence detected in PCR cycles 3 to 10. The results show an overall good correlation between the logarithm to the initial cDNA copy number and the Cₜ value. The correlation appears as a straight line with slope between −3.456 and −3.499 depending on the probe and correlation coefficients between 0.9981 and 0.9999. The slope of the curves reflect the efficiency of the PCRs with a 100% efficiency corresponding to a slope of −3.322 assuming a doubling of amplicon in each PCR cycle. The slopes of the present PCRs indicate PCR efficiencies between 94% and 100%. The correlation coefficients and the PCR efficiencies are as high as or higher than the values obtained with DNA 5′ nuclease assay probes 17 to 26 nucleotides long in detection assays of the same SSA4 cDNA levels (results not shown). Therefore these result show that the three 9-mer 5′ nuclease assay probes meet the requirements for true expression probes indicating that the probes should perform in expression profiling assays

### Example 8

### Detection of SSA4 Transcription Levels in Yeast

Expression levels of the SSA4 transcript were detected in different yeast strains grown at different culture conditions (±heat shock). A standard laboratory strain of Saccharomyces cerevisiae was used as wild type yeast in the experiments described here. A SSA4 knockout mutant was obtained from EUROSCARF (accession number Y06101). This strain is here referred to as the SSA4 mutant. Both yeast strains were grown in YPD medium at 30° C. till an OD₆₀₀ of 0.8 A. Yeast cultures that were to be heat shocked were transferred to 40° C. for 30 minutes after which the cells were harvested by centrifugation and the pellet frozen at −80° C. Non-heat shocked cells were in the meantime left growing at 30° C. for 30 minutes and then harvested as above.

RNA was isolated from the harvested yeast using the FastRNA Kit (Bio 101) and the FastPrep machine according to the supplier's instructions.

Reverse transcription was performed with 5 μg of anchored oligo(dT) primer to prime the reaction on 1 μg of total RNA, and 0.2 U of the reverse transcriptase Superscript II RT (Invitrogen) according to the suppliers instructions except that 20 U Superase-In (RNAse inhibitor—Ambion) was added. After a two-hour incubation, enzyme inactivation was performed at 70° for 5 minutes. The cDNA reactions were diluted 5 times in 10 mM Tris buffer pH 8.5 and oligonucleotides and enzymes were removed by purification on a MicroSpin™ S-400 HR column (Amersham Pharmacia Biotech). Prior to performing the expression assay the cDNA was diluted 20 times. The expression assay was performed with the Dual-labelled-570 probe using standard 5′ nuclease assay conditions except 2 μL of template was added. The template was a 100 times dilution of the original reverse transcription reactions. The four different cDNA templates used were derived from wild type or mutant with or without heat shock. The assay produced the expected results showing increased levels of the SSA4 transcript in heat shocked wild type yeast (Cₜ=26.1) compared to the wild type yeast that was not submitted to elevated temperature (Cₜ=30.3). No transcripts were detected in the mutant yeast irrespective of culture conditions. The difference in Cₜ values of 3.5 corresponds to a 17 fold induction in the expression level of the heat shocked versus the non-heat shocked wild type yeast and this value is close to the values around 19 reported in the literature (Causton, et al. 2001). These values were obtained by using the standard curve obtained for the Dual-labelled-570 probe in the quantification experiments with known amounts of the SSA4 transcript. The experiments demonstrate that the 9-mer probes are capable of detecting expression levels that are in good accordance with published results.

### Example 9

### Multiple Transcript Detection With Individual 9-mer Probes

To demonstrate the ability of the three 5′ nuclease assay probes to detect expression levels of other genes as well, three different yeast genes were selected in which one of the probe sequences was present. Primers were designed to amplify a 60-100 base pair region around the probe sequence. The three selected yeast genes and the corresponding primers are shown in Table 5.

TABLE 5
Design of alternative expression assays (SEQ ID NOs: 18-23)

| | | Forward primer | Reverse primer | Amplicon |
|---|---|---|---|---|
| Sequence/Name | Matching Probe | sequence | sequence | length |
| YEL055C/POL5 | Dual-labelled- | gcgagagaaaacaagca | attcgtcttcactggc | 94 bp |
| | 469 | agg | atca | |
| YDL149W_APG9 | Dual-labelled- | cagctaaaaatgatgac | attacatcatgattag | 97 bp |
| | 570 | aataatgg | ggaatgc | |
| YPL240C_HSP82 | Dual-labelled- | gggtttgaacattgatg | ggtgtcagctggaacc | 88 bp |
| | 671 | agga | tctt | |

Total cDNA derived from non-heat shocked wild type yeast was used as template for the expression assay, which was performed using standard 5′ nuclease assay conditions except 2 μL of template was added. All three probes could detect expression of the genes according to the assay design outlined in Table 5. Expression was not detected with any other combination of probe and primers than the ones outlined in Table 5. Expression data are available in the literature for the SSA4, POL5, HSP82, and the APG9 (Holstege, et al. 1998). For non-heat shocked yeast, these data describe similar expression levels for SSA4 (0.8 transcript copies per cell), POL5 (0.8 transcript copies per cell) and HSP82 (1.3 transcript copies per cell) whereas APG9 transcript levels are somewhat lower (0.1 transcript copies per cell).

These data are in good correspondence with the results obtained here since all these genes showed similar Cₜ values except HSP82, which had a Cₜ value of 25.6. This suggests that the HSP82 transcript was more abundant in the strain used in these experiments than what is indicated by the literature. The agarose gel shows that PCR product was indeed generated in reactions where no signal was obtained and therefore the lack fluorescent signal from these reactions was not caused by failure of the PCR. Furthermore, the different length of amplicons produced in expression assays for different genes indicate that the signal produced in expression assays for different genes are indeed specific for the gene in question.

### Example 10

### The General Experimental Procedure Related to Extendable Probes

The general structure of the dual-labelled probes is 5 ′-Fitc-dₘL¹L²L³L⁴L⁵L⁶L⁷Qdₙ-3′, where dₘ and dₙ designates an oligomer consisting of n natural nucleosides (a, g, c, t) and where n is an integer of from 1 to 20;

L¹ through L⁷ designates an oxy-LNA nucleotide or one or more of L¹ through L⁷ is X, where X designates an amino-LNA-group, attached to a quencher.

Optionally one or more natural nucleosides is/are interspersed in the oxy-LNA nucleotide sequence.

Primer extension was performed with extendable probes on synthetic oligonucleotide templates using heat-stable DNA polymerase (HotStarTaq, Qiagen) and 40 cycles of annealing and extension similar to conditions used for qPCR.

The final concentration of probe and template was 0.2 μM prior to thermocycling. The relative high concentration of the oligonucleotide template was used to increase the yield of the extension product, which is expected to be low due to the linear amplification nature of primer extension reactions compared to the exponential amplification in PCR.

To increase the sensitivity of detection 0.1 μCi of α-³²P-dCTP (Amersham Biosciences) was included in all primer extension reactions. Primer extension products were separated on 15% TBE-Urea gels (Invitrogen) and analysed for FITC-fluorescence using a Typhoon Imager (Amersham Biosciences). Gels were then stained in GelStar (Cambrex) and re-analysed on the Typhoon Imager. Finally gels were exposed for storage phosphor screen for detection of radioactive-labelled extension products.

### Example 11

### Extendable Probes in Primer Extension Reactions

The following probes (SEQ ID NOs: 24-33) were synthesized as described in Example 1.

| | |
|---|---|
| Probe no. | Composition |

| | |
|---|---|
| EQ#16215 | 5′-Fitc-cTGCCTCTQ1ttcctctg-3′ |
| EQ#16216 | 5′-Fitc-cTGCCTCTQ1ttc-3′ |
| EQ#16221 | 5′-Fitc-cTGCCTCTttcctctg-3′ |
| EQ#16222 | 5′-Fitc-cTGCCTCTttc-3′ |
| EQ#16224 | 5′-Fitc-cTGCCTCTttcctctg-P-3′ |
| EQ#16225 | 5′-Fitc-cTGCCTCTttc-P-3′ |
| EQ#16435 | 5′-Fitc-cTGCCTCXttcctctg-3′ |
| EQ#16340 | 5′-Fitc-cTGCCTCXttc-3′ |
| EQ#16342 | 5′-Fitc-cTGCCXCTttcctctg-3′ |
| EQ#16343 | 5′-Fitc-cTGCCXCTttc-3′ |

Fitc is fluorescein (6-FITC (Glenn Research, Prod. Id. No. 10-1964)).

Upper case (A, T, G, C) designates oxy-LNA.

A, T and G designates oxy-LNA substituted with one of the bases adenine, thymine or guanine, whereas C designates the base 5-methyl-cytosine.

Lower case (a, t, c, g ) designates natural nucleosides.

P designates a phosphate group.

X designates an amino-LNA nucleotide attached to a Dabcyl quencher (4-((4-(dimethylamino)phenyl)azo)benzoic acid, succinimidyl ester, Molecular Probes/Invitrogen).

Q1 designates the quencher prepared as described in Example 15.

The Synthetic Templates Used Are.

| | |
|---|---|
| EQ#15912 | |
| 5′-gtggtcgaaagcaatggacttgcaggaggagca | (SEQ ID NO:34) |
| gaggaaagaggcagaaggagaagcccataccaaggg | |
| ttcgaatccc-3′ | |
| EQ#16234 | |
| 5′-gtggtcgaaagcaatggacttgcaggaggagca | (SEQ ID NO:35) |
| gaggaaagaggcagaaggagaagcccataccaaggg | |
| ttcgaatccc-P-3′. | |

Reaction Conditions (Final Concentrations) in 50 μL Total Volume

| | |
|---|---|
| Template | 0.2 μM |
| Probe | 0.2 μM |
| HotStarTaq buffer | 1× |
| Mg²⁺ | 4 mM |
| dNTP's | 200 μM dATP, dGTP, dTTP and 20 μM dCTP |
| ³²P-dCTP | 0.02 μCi/μL |
| HotStarTaq | 0.05 U/μL |

PCR Cycler Settings

10 min 95° C.

40 cycles of (20 sec at 95° C. followed by 1 min at 60° C.)

on hold at 4° C.

Experiment I

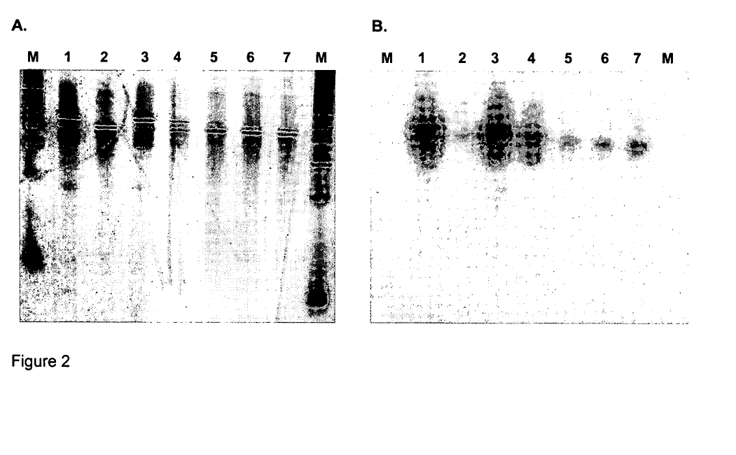

The results of primer extension experiments with EQ#16215, 16216, 16221, 16222, 16224, and 16225 are shown in FIGS. 2A and 2B (A, gel stained for nucleic acids with GelStar and B, autoradiography of the same gel). M represents molecular size marker lane and lane 1-6 contain extension reactions for probes EQ#16215, 16216, 16221, 16222, 16224, and 16225, respectively; lane 7 contains extension reaction for template without probe.

As expected template alone (lane 7) does not sustain incorporation of radioactivity, the same is true for template in combination with probes blocked by a phosphate molecule in the 3′-end to prevent extension (lane 5-6). Probes containing 7 LNA nucleotides, a quencher, followed by 8 standard DNA nucleotides in the 3′-end are extendable irrespective of the presence of a quencher (lane 1 and 3). If no quencher is present, a probe containing 7 LNA nucleotides followed by only 3 standard DNA nucleotides is clearly extendable (lane 4).

Experiment II

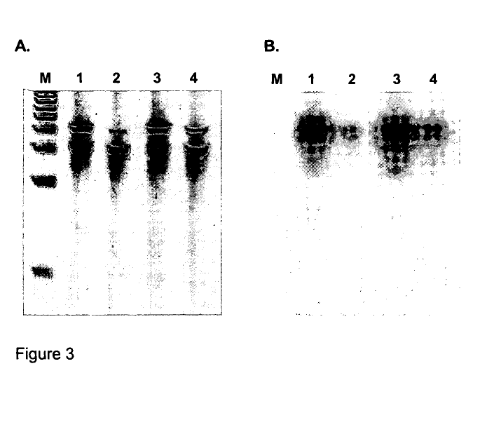

The results of primer extension experiments with EQ#16435, 16340, 16342, and 16343 are shown in FIGS. 3A and 3B (A, gel stained for nucleic acids with GelStar and B, autoradiography of the same gel). M represents molecular size marker lane and lane 1-4 contain extension reactions for probes EQ#16435, 16340, 16342, and 16343, respectively.

As in experiment I probes containing 7 LNA nucleotides, a quencher, followed by 8 standard DNA nucleotides in the 3′-end are extendable in the presence of a quencher (lane 1 and 3). Template alone does not sustain incorporation of radioactivity. In this experiment the quencher is attached to an amino-LNA-T residue and in contrast to experiment I this supports extension from a probe containing 7 LNA nucleotides, followed by only 3 standard DNA nucleotides (lane 2). If the quencher is attached to an amino-LNA-T residue within the block of LNA residues, the probe is still extendable (lane 4).

### Example 12

### Using Extendable Probes Containing a Block of LNA Monomers in a PCR Reaction

To demonstrate that Extendable Probes containing a block of LNA monomers do not function as template for the polymerase reaction extending the reverse PCR primer, the following experiment was performed:

For the experiments artificial oligonucleotide target EQ#16234 was used, where the 3′-end is phosphorylated to prevent unintended extension.

A DNA primer was used for PCR amplification with the following sequence (SEQ ID NO: 36):

| | |
|---|---|
| EQ#15910 | 5′-gtggtcgaaagcaatggact-3′ |

An Extendable Probe with the following sequence (SEQ ID NO: 37) was used:

EQ#16214
5′-Fluorescein-cTGCCTCT-Q1-ttcctctgctcctcct-3′

Upper case letters denoting LNA monomers, lower case letters denoting DNA monomers. Q1 is a quencher moiety (Prepared as described in Example 15).

Reagents for PCR amplification were mixed according to the following scheme in 50 μL final reaction volume:

| Reagents | Final Concentration | |
|---|---|---|
| H₂O | | |
| Qiagen 10× PCR buffer | 1× | |
| Mg²⁺ | 4.0 | mM |
| dNTP | 0.2 | mM |
| Extendable Probe | 0.2 | μM |
| Oligonucleotide Template | 4 | pM |
| EQ#15910 | 0.9 | μM |
| Qiagen Hot Star Taq | 0.05 | U/μL |

| | |
|---|---|
| ROX Reference Dye | 0.1× |
| (Invitrogen) | |

PCR was performed in a PRISM 7500 (ABI) using the following PCR cycle protocols:

| | |
|---|---|
| Hot Start: | 95° C. for 10 minutes |
| Amplification for 40 cycles: | 94° C. for 20 seconds |
| | 60° C. for 1 minute |

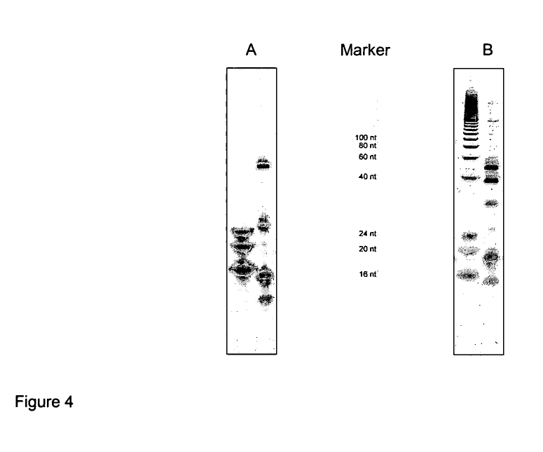

After PCR amplification the reaction mixture was analysed by gel electrophoresis on a 15% TBE-Urea pre-cast Novex gel. An aliquot of the reaction mixture was mixed 1:1 with TBE-Urea loading buffer containing glycerol and 10 μL was loaded on gel. As size marker “PCR Low Ladder, 20 bp” from Sigma mixed with 3′ fluorescein labelled oligos of 16 nt, 20 nt and 24 nt respectively was used (approx 25 nM each). The gel electrophoresis was performed at 180 V constant voltage for 50 min with 1×TBE as the running buffer. The gel was scanned in a Typhoon gel scanner, using the “Fluorescein”-channel and a PMT gain setting of 600V. Subsequently the gel was stained with GelStar solution (1:10.000 in TBE) for 5 min and scanned in the Typhoon again, using the same settings. FIGS. 4A and 4B show the gel scanned in the Fluorescein-channel immediately after electrophoresis (4A), and subsequent to GelStar staining (4B).

As it appears from the right lane of the Fluorescein image in the FIGS. 4A and 4B above, the PCR reaction results in a single sharp band that gives rise to a signal in the fluorescein channel. In the left lane the marker only gives rise to a fluorescent signal from the 3 fluorescein labelled oligos of 16 nt, 20 nt and 24 nt. When the gel is stained with GelStar a second sharp band appears in the right lane and which is approx 10 nt shorter in length. By comparing to the marker lane the original band appears to be between 40 nt and 60 nt, whereas the shorter band is a little shorter than 40 nt.

The expected product size of the extension product from extension of the Extendable Probe is 47 nt (including a block of LNA), and the expected extension product size from extension of the reverse primer (EQ#15910) is 39 nt, provided that the polymerase cannot use the LNA-block as template.

### Example 13

### Effect of Shortening the 3′-DNA Stretch in Extendable Probes Containing a Block of LNA Monomers, When Used in a PCR Reaction

An experiment was performed using 3 different extendable probes in a PCR reaction. The three different probes have a 3′-DNA-stretch of 16 nt, 8 nt and 3 nt, respectively of which the latter two DNA-stretches are considerably shorter than what would be expected to function as primer in a standard PCR reaction.

For the experiments, artificial oligonucleotide target EQ#16234 was used, where the 3′-end is phosphorylated to prevent unintended extension.

DNA primer EQ#15910 was used for PCR amplification. Extendable Probes EQ#16214, EQ#16221, and EQ#16222 were also used.

Reagents for PCR amplification were mixed according to the following scheme in 50 μL final reaction volume:

| Reagents | Final Concentration | |
|---|---|---|
| H₂O | | |
| Qiagen 10× PCR buffer | 1× | |
| Mg²⁺ | 4.0 | mM |
| dNTP | 0.2 | mM |
| Extendable Probe | 0.2 | μM |
| Oligonucleotide Template | 4 | pM |
| EQ#15910 | 0.9 | μM |
| Qiagen Hot Star Taq | 0.05 | U/μL |

| | |
|---|---|
| ROX Reference Dye | 0.1× |
| (Invitrogen) | |

PCR was performed in a PRISM 7500 (ABI) using the following PCR cycle protocols:

| | |
|---|---|
| Hot Start: | 95° C. for 10 minutes |
| Amplification for 40 cycles: | 94° C. for 20 seconds |
| | 60° C. for 1 minute |

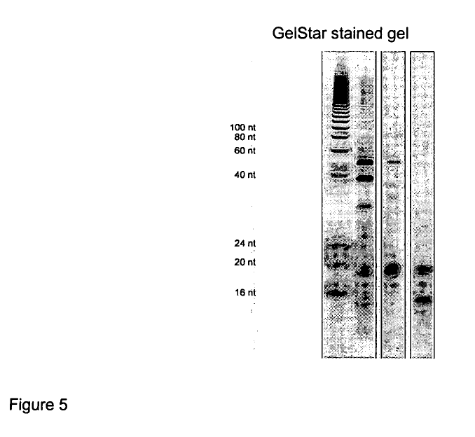

After PCR amplification the reaction mixture was analysed by gel electrophoresis on a 15% TBE-Urea pre-cast Novex gel. An aliquot of the reaction mixture was mixed 1:1 with TBE-Urea loading buffer containing glycerol and 10 μL was loaded on gel. As size marker was used “PCR Low Ladder, 20 bp” from Sigma mixed with 3 fluorescein labelled oligonucleotides of 16 nt, 20 nt and 24 nt respectively (approx 25 nM each). The gel electrophoresis was performed at 180 V constant voltage for 50 min with 1×TBE as the running buffer. The gel was scanned in a Typhoon gel scanner, using the “Fluorescein”-channel and a PMT gain setting of 600V. Subsequently the gel was stained with GelStar solution (1:10.000 in TBE) for 5 min and scanned in the Typhoon again, using the same settings. FIG. 5 shows the gel scanned in the Fluorescein-channel subsequent to GelStar staining. From left to right the lanes contain: DNA marker, EQ#16214, EQ#16221 and EQ#16222.

As it appears from FIG. 5 the PCR reaction gives rise to two sharp bands when using the extendable probe EQ#16214. When the extendable probe EQ#16221 is used the two bands are still visible (the shorter band having a slightly higher mobility due to the lack of the Q1 quencher moiety). When the Extendable Probe EQ#16222 is used none of the two bands are visible.

### Example 14

### The Use of Extendable Probes in Real-Time PCR

To demonstrate the functionality of Extendable Probes in real-time PCR the following experiment was performed using an Extendable Probe:

For this experiment, artificial oligonucleotide target EQ#16234 was used, where the 3′-end is phosphorylated to prevent extension.

Two primers were used for PCR amplification with the following sequences:

| | |
|---|---|
| EQ#15910 | |
| 5′-gtggtcgaaagcaatggact-3′ | (SEQ ID NO:38) |
| EQ#15911 | |
| 5′-gggattcgaacccttggtat-3′ | |

Extendable Probe EQ#16215 was used.

Reagents for the real-time PCR reaction were mixed according to the following scheme in 50 μL final reaction volume:

| Reagents | Final Concentration | |
|---|---|---|
| H₂O | | |
| Qiagen 10× PCR buffer | 1× | |
| Mg²⁺ | 4.0 | mM |
| dNTP | 0.2 | mM |
| Extendable Probe | 0.2 | μM |
| Oligonucleotide Template | 4 | pM |
| EQ#15910 | 0.9 | μM |
| EQ#15911 | 0.9 | μM |
| Qiagen Hot Star Taq | 0.05 | U/μL |

| | |
|---|---|
| ROX Reference Dye | 0.1× |
| (Invitrogen) | |

Real-time PCR was performed in a PRISM 7500 (ABI) using the following PCR cycle protocols:

| | |
|---|---|
| Hot Start: | 95° C. for 10 minutes |
| Amplification for 40 cycles: | 94° C. for 20 seconds |
| | 60° C. for 1 minute |
| | Fluorescence detection |

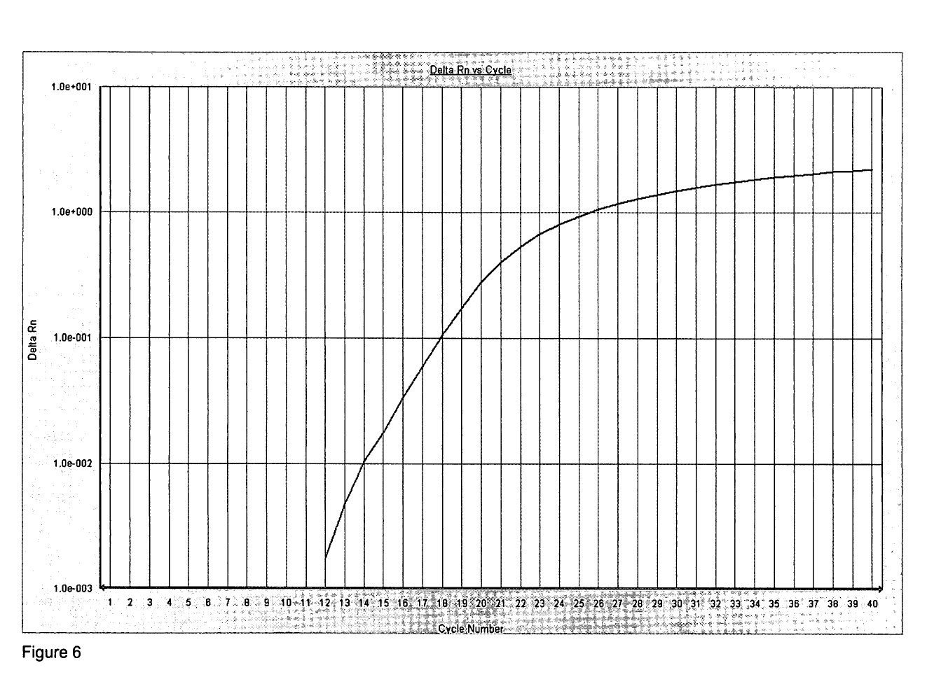

FIG. 6 is a screen dump from the qPCR instrument software showing that the Extendable Probe produced the expected increase in fluorescence intensity as a function of the number of amplification cycles.

### Example 15

### Preparation of the Quencher Q1 of the Formula 1-(3-(2-cyanoethoxy(diisopropylamino)phosphinoxy)propylamino)-4-(3-(4,4′-dimethoxy-trityloxy)propylamino)-anthraquinone (3)

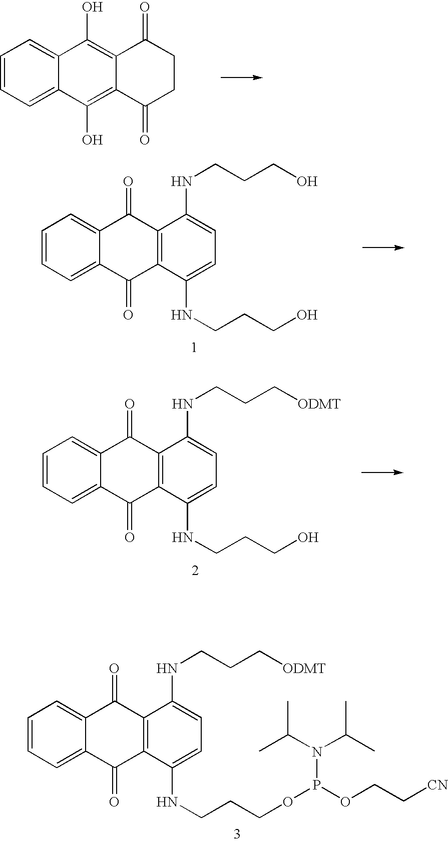

### 1,4-Bis(3-hydroxypropylamino)-anthraquinone (1)

Leucoquinizarin (9.9 g; 0.04 mol) is mixed with 3-amino-1-propanol (10 mL) and Ethanol (200 mL) and heated to reflux for 6 hours. The mixture is cooled to room temperature and stirred overnight under atmospheric conditions. The mixture is poured into water (500 mL) and the precipitate is filtered off washed with water (200 mL) and dried. The solid is boiled in ethylacetate (300 mL), cooled to room temperature and the solid is collected by filtration.

Yield: 8.2 g (56%)

### 1-(3-4,4′-dimethoxy-trityloxy)propylamino)-4-(3-hydroxypropylamino)-anthraquinone (2)

1,4-Bis(3-hydroxypropylamino)-anthraquinone (7.08 g; 0.02 mol) is dissolved in a mixture of dry N,N-dimethylformamide (150 mL) and dry pyridine (50 mL). Dimethoxytritylchloride (3.4 g; 0.01 mol) is added and the mixture is stirred for 2 hours. Additional dimethoxytritylchloride (3.4 g; 0.01 mol) is added and the mixture is stirred for 3 hours. The mixture is concentrated under vacuum and the residue is redissolved in dichloromethane (400 mL) washed with water (2×200 ml) and dried (Na₂SO₄). The solution is filtered through a silica gel pad (ø 10 cm; h 10 cm) and eluted with dichloromethane until mono-DMT-anthraquinone product begins to elude where after the solvent is the changed to 2% methanol in dichloromethane. The pure fractions are combined and concentrated resulting in a blue foam.

Yield: 7.1 g (54%)

¹H-NMR(CDCl₃): 10.8 (2H, 2xt, J=5.3 Hz, NH), 8.31 (2H, m, AqH), 7.67 (2H, dt, J=3.8 and 9.4, AqH), 7.4-7.1 (9H, m, ArH+AqH), 6.76 (4H, m, ArH) 3.86 (2H, q, J=5.5 Hz, CH₂OH), 3.71 (6H, s, CH₃), 3.54 (4H, m, NCH₂), 3.26 (2H, t, J=5.7 Hz, CH₂ODMT), 2.05 (4H, m, CCH₂C), 1.74 (1H, t, J=5 Hz, OH).

### 1-(3-(2-cyanoethoxy(diisopropylamino)phosphinoxy)propylamino)-4-(3-(4,4′-dimethoxy-trityloxy)propylamino)-anthraquinone (3)

1-(3-(4,4′-dimethoxy-trityloxy)propylamino)-4-(3-hydroxypropylamino)-anthraquinone (0.66 g; 1.0 mmol) is dissolved in dry dichloromethane (100 mL) and added 3 Å molecular sieves. The mixture is stirred for 3 hours and then added 2-cyanoethyl-N,N,N′,N′-tetraisopropylphosphordiamidite (335 mg; 1.1 mmol) and 4,5-dicyanoimidazole (105 mg; 0.9 mmol). The mixture is stirred for 5 hours and then added sat. NaHCO₃ (50 mL) and stirred for 10 minutes. The phases are separated and the organic phase is washed with sat. NaHCO₃ (50 mL), brine (50 mL) and dried (Na₂SO₄). After concentration the phosphoramidite is obtained as a blue foam and is used in oligonucleotide synthesis without further purification.

Yield: 705 mg (82%)

³¹ P-NMR (CDCl₃): 150.0

¹H-NMR(CDCl₃): 10.8 (2H, 2xt, J=5.3 Hz, NH), 8.32 (2H, m, AqH), 7.67 (2H, m, AqH), 7.5-7.1 (9H, m, ArH+AqH), 6.77 (4H, m, ArH) 3.9-3.75 (4H, m), 3.71 (6H, s, OCH₃), 3.64-3.52 (3.54 (6H, m), 3.26 (2H, t, J=5.8 Hz, CH₂ODMT), 2.63 (2H, t, J=6.4 Hz, CH₂CN) 2.05 (4H, m, CCH₂C), 1.18 (12H, dd, J=3.1 Hz, CCH₃).

### Other Embodiments

The description of the specific embodiments of the invention is presented for the purposes of illustration. It is not intended to be exhaustive nor to limit the scope of the invention to the specific forms described herein. Although the invention has been described with reference to several embodiments, it will be understood by one of ordinary skill in the art that various modifications can be made without departing from the spirit and the scope of the invention, as set forth in the claims. All patents, patent applications, and publications referenced herein are hereby incorporated by reference.

Other embodiments are within the claims.

What is claimed is:

1. A labelled oligonucleotide probe comprising a sequence complementary to a region of a target nucleic acid sequence,
wherein said labelled oligonucleotide probe is extendable by a polymerase to allow incorporation of said labelled oligonucleotide probe into a primer extension product and wherein the replication of all or part of said labelled oligonucleotide probe by a polymerase is prevented.

2. A probe of claim 1, wherein the replication by a polymerase of all or part of said labelled oligonucleotide probe is blocked by the presence in the probe of a moiety which inhibits the replication.

3. A probe of claim 2, wherein said moiety is a LNA, an MGB, a HEG, an intercalator, an INA, an ENA, a dye, or a quencher.

4. A probe of claim 2, wherein the moiety is a linker connecting two oligonucleotide sequences.

5. A probe of claim 1, wherein the complement of a part of said labelled oligonucleotide probe is capable of being a template for said labelled oligonucleotide probe in a PCR reaction.

6. A probe of claim 1, wherein the complement of a part of the 3′ end of said labelled oligonucleotide probe is capable of being a template for said labelled oligonucleotide probe in a PCR reaction.

7. A probe of claim 1, wherein no more than eight nucleotides at the 3′ end of said labelled oligonucleotide probe are capable of being replicated.

8. A probe of claim 7, wherein no more than five nucleotides at the 3′ end of said labelled oligonucleotide probe are capable of being replicated.

9. A probe of claim 8, wherein no more than three nucleotides at the 3′ end of said labelled oligonucleotide probe are capable of being replicated.

10. A probe of claim 1, wherein at least a part of said labelled oligonucleotide probe cannot act as a template for polymerase replication in a reaction which otherwise is capable of generating partially or entirely complementary target sequences for said labelled oligonucleotide probe.

11. A probe of claim 1, wherein at least a part of said labelled oligonucleotide probe cannot act as a template for polymerase replication in a reaction which otherwise is capable of generating a complementary part of said labelled oligonucleotide probe sufficient to act as template for said labelled oligonucleotide probe in a PCR reaction.

12. A probe of claim 1, wherein a substantial part of the 3′ end of said labelled oligonucleotide probe cannot act as a template for polymerase replication in a reaction which otherwise is capable of generating additional partially or entirely complementary probe target sequences sufficient to act as template for said labelled oligonucleotide probe in a PCR reaction.

13. A polymerase chain reaction (PCR) amplification process for detecting a target nucleic acid sequence in a sample, said process comprising:
(a) contacting said sample with at least one labelled oligonucleotide probe of claim 1 and a first oligonucleotide primer comprising a sequence complementary to a region in one strand of the target nucleic acid sequence and priming the synthesis of a complementary DNA strand, wherein said first oligonucleotide primer anneals to its complementary region upstream of any labelled oligonucleotide probe annealed to the same nucleic acid strand;
(b) amplifying the target nucleic acid sequence using a nucleic acid polymerase having 5′ to 3′ nuclease activity as a template-dependent polymerizing agent under conditions which are permissive for PCR cycling steps of (i) annealing of said first oligonucleotide primer and said labelled oligonucleotide probe to a template nucleic acid sequence contained within the target sequence, and (ii) extending the first oligonucleotide primer wherein said nucleic acid polymerase synthesizes a primer extension product while the 5′ to 3′ nuclease activity of the nucleic acid polymerase simultaneously releases labelled fragments from the annealed duplexes comprising the labelled oligonucleotide probe and its complementary template nucleic acid sequence, thereby creating detectable labelled fragments; and
(c) detecting the presence or absence of labelled fragments to determine the presence or absence of the target sequence in said sample.

14. The process of claim 13, wherein step (a) further comprises contacting said sample with a second oligonucleotide primer comprising a sequence complementary to a region in the second strand of the target nucleic acid sequence and priming the synthesis of a complementary DNA strand, and wherein in step (b) said labelled oligonucleotide probe anneals to the target nucleic acid sequence bounded by the first and second oligonucleotide primers.

15. The process of claim 13, wherein the labelled oligonucleotide probe comprises a pair of labels effectively positioned to generate a detectable signal, said labels being separated by a site within the oligonucleotide probe that is cleaved by the 5′ to 3′ nuclease activity of the nucleic acid polymerase in step (b)(ii).

16. The process of claim 13, wherein the labelled oligonucleotide probe comprises a pair of labels effectively positioned to quench the generation of detectable signal, said labels being separated by a site within the oligonucleotide probe that is cleaved by the 5′ to 3′ nuclease activity of the nucleic acid polymerase in step (b)(ii).

17. A library comprising a plurality of labelled oligonucleotide probes of claim 1, wherein each probe in the library comprises a recognition sequence tag and a detection moiety, wherein at least one monomer in each oligonucleotide probe is a modified monomer analogue, increasing the binding affinity for the complementary target sequence relative to the corresponding unmodified oligodeoxyribonucleotide, such that the probes have sufficient stability for sequence-specific binding and detection of a substantial fraction of a target nucleic acid in any given target population.

18. The library of claim 17, wherein the number of different recognition sequences comprises less than 10% of all possible sequence tags of a given length(s).


SEQUENCE LISTING (38 sequences)

SEQ ID NO: 1 - DNA, 13 bp, Artificial Sequence
  Synthetic
  modified_base 1-13: LNA substituted with one of the bases adenine, methyl-cytosine, thymine or guanine
  modified_base 1: Fluorescein is attached to position 1
  modified_base 13: Dabcyl quencher is attached to position 13
caaggagaagttg

SEQ ID NO: 2 - DNA, 12 bp, Artificial Sequence
  Synthetic
  modified_base 1-9, 12: LNA substituted with one of the bases adenine, guanine, methyl-cytosine, or thymine.
  modified_base 1: Fluorescein is attached to position 1
  modified_base 12: Dabcyl quencher is attached to position 12
caaggaaagttg

SEQ ID NO: 3 - DNA, 23 bp, Artificial Sequence
  Synthetic
cgcgtttactttgaaaaattctg

SEQ ID NO: 4 - DNA, 20 bp, Artificial Sequence
  Synthetic
gcttccaatttcctggcatc

SEQ ID NO: 5 - DNA, 25 bp, Artificial Sequence
  Synthetic
gcccaagatgctataaattggttag

SEQ ID NO: 6 - DNA, 23 bp, Artificial Sequence
  Synthetic
gggtttgcaacaccttctagttc

SEQ ID NO: 7 - DNA, 18 bp, Artificial Sequence
  Synthetic
tacggagctgcaggtggt

SEQ ID NO: 8 - DNA, 18 bp, Artificial Sequence
  Synthetic
gttgggccgttgtctggt

SEQ ID NO: 9 - DNA, 20 bp, Artificial Sequence
  Synthetic
gcgagagaaaacaagcaagg

SEQ ID NO: 10 - DNA, 20 bp, Artificial Sequence
  Synthetic
attcgtcttcactggcatca

SEQ ID NO: 11 - DNA, 25 bp, Artificial Sequence
  Synthetic
cagctaaaaatgatgacaataatgg

SEQ ID NO: 12 - DNA, 23 bp, Artificial Sequence
  Synthetic
attacatcatgattagggaatgc

SEQ ID NO: 13 - DNA, 21 bp, Artificial Sequence
  Synthetic
gggtttgaacattgatgagga

SEQ ID NO: 14 - DNA, 20 bp, Artificial Sequence
  Synthetic
ggtgtcagctggaacctctt

SEQ ID NO: 15 - DNA, 35 bp, Artificial Sequence
  Synthetic
acgtgagctcattgaaactgcaggtggtattatga

SEQ ID NO: 16 - DNA, 44 bp, Artificial Sequence
  Synthetic
gatccccgggaattgccatgctaatcaacctcttcaaccgttgg

SEQ ID NO: 17 - DNA, 50 bp, Artificial Sequence
  Synthetic
acgtggatccttttttttttttttttttttgatccccgggaattgccatg

SEQ ID NO: 18 - DNA, 20 bp, Artificial Sequence
  Synthetic
gcgagagaaaacaagcaagg

SEQ ID NO: 19 - DNA, 20 bp, Artificial Sequence
  Synthetic
attcgtcttcactggcatca

SEQ ID NO: 20 - DNA, 25 bp, Artificial Sequence
  Synthetic
cagctaaaaatgatgacaataatgg

SEQ ID NO: 21 - DNA, 23 bp, Artificial Sequence
  Synthetic
attacatcatgattagggaatgc

SEQ ID NO: 22 - DNA, 21 bp, Artificial Sequence
  Synthetic
gggtttgaacattgatgagga

SEQ ID NO: 23 - DNA, 20 bp, Artificial Sequence
  Synthetic
ggtgtcagctggaacctctt

SEQ ID NO: 24 - skipped
  synthetic

SEQ ID NO: 25 - skipped
  synthetic

SEQ ID NO: 26 - DNA, 16 bp, Artificial Sequence
  Synthetic
  modified_base 1: Fluorescein is attached to position 1
  modified_base 2, 3, 6, 8: oxy-LNA substituted with one of the bases adenine, thymine or guanine
  modified_base 4, 5, 7: oxy-LNA substituted with 5-methyl-cytosine
ctgcctctttcctctg

SEQ ID NO: 27 - DNA, 11 bp, Artificial Sequence
  Synthetic
  modified_base 1: Fluorescein is attached to position 1
  modified_base 2, 3, 6, 8: oxy-LNA substituted with one of the bases adenine, thymine or guanine
  modified_base 4,5,7: oxy-LNA substituted with 5-methyl-cytosine
ctgcctctttc

SEQ ID NO: 28 - DNA, 16 bp, Artificial Sequence
  Synthetic
  modified_base 1: Fluorescein is attached to  position 1
  modified_base 16: Position 16 is phosphorylated
  modified_base 2,3,6,8: oxy-LNA substituted with one of the bases adenine, thymine or guanine
  modified_base 4,5,7: oxy-LNA substituted with base 5-methyl-cytosine
ctgcctctttcctctg

SEQ ID NO: 29 - DNA, 11 bp, Artificial Sequence
  Synthetic
  modified_base 1: Fluorescein is attached to position 1
  modified_base 11: Position 11 is phosphorylated
  modified_base 2,3,6,8: oxy-LNA substituted with one of the bases adenine, thymine or guanine
  modified_base 4,5,7: oxy-LNA substituted with base 5-methyl-cytosine
ctgcctctttc

SEQ ID NO: 30 - DNA, 16 bp, Artificial Sequence
  Synthetic
  modified_base 1: Fluorescein is attached to position 1
  misc_feature 8: n = amino-LNA nucleotide attached to a Dabcyl Quencher
  modified_base 2,3,6: oxy-LNA substituted with one of the bases adenine, thymine or guanine
  modified_base 4,5,7: oxy-LNA substituted with base 5-methyl-cytosine
ctgcctcnttcctctg

SEQ ID NO: 31 - DNA, 11 bp, Artificial Sequence
  Synthetic
  modified_base 1: fluorescein is attached to position 1
  misc_feature 8: n = amino-LNA nucleotide attached to a Dabcyl Quencher
  modified_base 2,3,6: oxy-LNA substituted with one of the bases adenine, thymine or guanine
  modified_base 4,5,7: oxy-LNA substituted with 5-methyl-cytosine
ctgcctcnttc

SEQ ID NO: 32 - DNA, 16 bp, Artificial Sequence
  Synthetic
  modified_base 1: fluorescein is attached to position 1
  misc_feature 6: n = amino-LNA nucleotide attached to a Dabcyl Quencher
  modified_base 2, 3, 8: oxy-LNA substituted with one of the bases adenine,thymine or guanine
  modified_base 4, 5, 7: oxy-LNA substituted with 5-methyl-cytosine
ctgccnctttcctctg

SEQ ID NO: 33 - DNA, 11 bp, Artificial Sequence
  Synthetic
  modified_base 1: fluorescein is attached to position 1
  misc_feature 6: n = amino-LNA-nucleotide attached to a Dabcyl Quencher
  modified_base 2,3,8: oxy-LNA substituted with one of the bases adenine, thymine or guanine
  modified_base 4,5,7: oxy-LNA substituted with 5-methyl-cytosine
ctgccnctttc

SEQ ID NO: 34 - DNA, 79 bp, Artificial Sequence
  Synthetic
gtggtcgaaagcaatggacttgcaggaggagcagaggaaagaggcagaaggagaagcccataccaagggttcgaatccc

SEQ ID NO: 35 - DNA, 79 bp, Artificial Sequence; identical to SEQ ID NO: 34
  Synthetic

SEQ ID NO: 36 - DNA, 20 bp, Artificial Sequence
  Synthetic
gtggtcgaaagcaatggact

SEQ ID NO: 37 - DNA, 16 bp, Artificial Sequence
  Synthetic
  modified_base 1: Quencher Q1 is attached at position 1
ttcctctgctcctcct

SEQ ID NO: 38 - DNA, 20 bp, Artificial Sequence
  Synthetic
gggattcgaacccttggtat